(19) United States
(12) Patent Application Publication
(10) Pub. No.: US 2024/0382717 A1
(43) Pub. Date: Nov. 21, 2024

(54) STEERABLE TUBULAR ASSEMBLY FOR BRONCHOSCOPIC PROCEDURES

(71) Applicant: W Endoluminal Robotics Ltd., Ariel (IL)

(72) Inventors: Eli SHAPIRA, Kefar Sava (IL); Tsahi Itshak GRIMBERG, Kefar Sava (IL); Nadav PAZ, Jerusalem (IL); Ron KAPPEL, Petah Tikva (IL)

(21) Appl. No.: 18/693,147

(22) PCT Filed: Sep. 4, 2022
(86) PCT No.: PCT/IB2022/058307

§ 371 Date: Mar. 18, 2024

Related U.S. Application Data
(60) Provisional application No. 63/247,424, filed on Sep. 23, 2021.

(51) Int. Cl. A61M 25/01

(52) U.S. Cl. CPC A61M 25/0138; A61M 25/0147; A61M 2025/015

(57) ABSTRACT

Apparatus (100) for facilitating a procedure on a subject, the apparatus comprising a catheter that comprises a head (131); a tube (102) comprising a proximal region (106), coupled to the head; a steering region (110), distal from the proximal region, and dimensioned for advancement into the subject; a bearing surface (266, 268) at the steering region; and a circumferential wall (104) extending from the proximal region to the steering region; and a wire (140, 180), wherein the wire: extends, from the head, distally to the bearing surface, is slidable over the bearing surface, curves around the bearing surface and proximally away from the bearing surface, and has a terminus (158) that is anchored to the tube at an anchoring location (162). Other embodiments are also described.

### CROSS-REFERENCES TO RELATED APPLICATIONS

The present application claims priority to Provisional U.S. Patent Application 63/247,424 to Shapira et al., filed Sep. 23, 2021, and titled “TUBULAR ASSEMBLY FOR BRONCHOSCOPIC PROCEDURES”, which is incorporated herein by reference.

### FIELD OF THE INVENTION

The present disclosure relates in general to an apparatus including a tubular assembly for medical procedures. More specifically, the present disclosure relates to a tubular assembly for advancement and steering into a subject during bronchoscopy.

### BACKGROUND

Steerable tubes, such as catheters, are routinely used to access body cavities of patients. However, it can be challenging to configure such steerable tubes for steering and advancement through particular anatomical lumens, such as airways.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Steering a catheter within a tortuous anatomical lumen may be performed by tensioning one or more wires, e.g., pullwires, adapted to bend and/or straighten the catheter while steering and advancing through the anatomical lumens. In some instances, a substantially large magnitude of tensioning force is required to apply on the pullwire for steering the wire.

A conventional steerable catheter pullwire typically extends in a simple longitudinal manner, from a proximal end of the catheter to a steerable distal region of the catheter, to which it is secured. In contrast, the present disclosure includes a tube (e.g., a catheter) that has a pullwire that, within a steering region of the tube, is arranged in a force-multiplication arrangement in which the wire defines multiple longitudinal segments separated by curved segments. For some applications this force-multiplication arrangement may be considered to have a similar effect as a conventional pulley (e.g., movable pulley) arrangement. The force-multiplication arrangement results in the tensioning force applied to the pullwire being multiplied within the steering region of the catheter (e.g., solely within the steering region of the catheter), e.g., with each of the longitudinal segments being subjected to a tensioning force equivalent to the aforementioned applied tensioning force. This arrangement significantly reduces the tensioning force required to be applied for steering the catheter.

In some applications, the pullwire may define more than two longitudinal segments within the steering region. In some applications, the pullwire may define more than one curved segment within the steering region.

In some applications, the catheter may be steered by a single pullwire. In some applications, a given steering region may have multiple such pullwires at different circumferential positions around the tube. In some applications, one of these multiple pullwires may be disposed closer to the channel of the tube than another of the multiple pullwires.

In some applications, the tube may be steered by a first pullwire operable for bending the tube and a second pullwire operable for straightening the tube.

In some applications, the steerable tube may have multiple steering regions at different axial positions along the tube.

In some applications, the catheter is a tube having a proximal region, an intermediate region, and a distal region. The distal region can be a steering region. In some applications the curved segment of the pullwire is disposed at the distal region.

In some applications, the pullwire is arranged to have three longitudinal segments. Depending on the arrangement of the curved and longitudinal segments, the mechanical advantage may be increased further, e.g., such that the degree of force multiplication is threefold, fourfold or more.

In some applications, the steering region has a distal segment and a proximal segment—e.g., with the wire being configured to confer a different mechanical advantage on the distal portion compared with the proximal portion.

In some such applications, the anchoring location is at a margin between the distal segment and the proximal segment. In some applications, the distal and proximal segments are equal lengths. In some applications, either the distal or the proximal segment has a longer length than the other.

In some such applications, the wire comprises three longitudinal segments—e.g., with the first and second longitudinal segments being present in both the proximal segment and the distal segment, but with the third longitudinal segment being absent from the distal segment.

In some other such applications, the wire comprises two longitudinal segments—e.g., with the first and second longitudinal segments being present in the distal segment, but with the second longitudinal segment being absent from the proximal segment.

In some applications, the terminus of the wire is anchored to the tube in a manner that the wire extends proximally to the terminus. In some applications, the terminus of the wire is anchored to the tube in a manner that the wire extends distally to the terminus.

There is therefore provided, in accordance with some applications, apparatus for facilitating a procedure on a subject, the apparatus including a catheter that includes a head, and/or a tube including: a proximal region, coupled to the head; a steering region, distal from the proximal region, and/or dimensioned for advancement into the subject.

In some applications, the tube may further include a bearing surface at the steering region; and/or a circumferential wall extending from the proximal region to the steering region; and/or a wire.

In some applications, the wire may extend, from the head, distally to the bearing surface, is slidable over the bearing surface, curves around the bearing surface and proximally away from the bearing surface, and/or has a terminus that is anchored to the tube at an anchoring location.

For some applications, the wire defines: a first longitudinal segment between the head and the bearing surface, and/or a second longitudinal segment between the bearing surface and the terminus, and/or within the steering region, the first longitudinal segment is substantially parallel with the second longitudinal segment.

For some applications, the wire extends proximally away from the bearing surface to the terminus.

For some applications, the steering region is a distal region of the tube.

For some applications, at the steering region, the wire is arranged in a force-multiplication arrangement that, upon application of a tensioning force to a proximal end of the wire, multiplies the tensioning force within the steering region to apply a bending force that bends the steering region.

For some applications, the steering region is no more flexible than the proximal region.

For some applications, the tube may extend from the proximal region to the steering region to define a longitudinal axis of the tube, and/or in response to application of a tensioning force to a proximal end of the wire, at the bearing surface the wire slides around the longitudinal axis.

For some applications, the tube extends from the proximal region to the steering region to define a longitudinal axis of the tube, and/or in response to application of a tensioning force to a proximal end of the wire, at the bearing surface the wire slides around a transverse axis that is transverse to the longitudinal axis.

For some applications, the tube extends from the proximal region to the steering region to define a longitudinal axis of the tube, and/or in response to application of a tensioning force to a proximal end of the wire, at the bearing surface the wire slides around the longitudinal axis.

For some applications, the tube is configured to be advanced through an anatomical lumen.

For some applications, the tube is configured to be advanced through an airway duct.

For some applications, the circumferential wall circumscribes, and thereby defines, a channel of the tube.

For some applications, the channel is configured to facilitate passage of a tool therethrough.

For some applications, the channel is configured to facilitate passage of an imaging device therethrough.

For some applications, the wire at least partially circumscribes the channel.

For some applications, at the bearing surface, the wire circumscribes the channel in an arc of 330-360 degrees.

For some applications, at the bearing surface, the wire circumscribes the channel in an arc of 330 degrees or less.

For some applications, at the bearing surface, the wire circumscribes the channel in an arc of 180 degrees or less.

For some applications, at the bearing surface, the wire circumscribes the channel in an arc of 90 degrees or less.

For some applications:

the bearing surface is a first bearing surface,

the tube includes a second bearing surface, and/or

the wire:
extends, from the first bearing surface, proximally to the second bearing surface, and/or
is slidable over the second bearing surface, and/or
curves around the second bearing surface and distally away from the second bearing surface.

For some applications, the wire extends distally to the terminus.

For some applications, the anchoring location is located adjacent the first bearing surface.

For some applications, the anchoring location is located adjacent the second bearing surface.

For some applications, the anchoring location is located partway between the first bearing surface and the second bearing surface.

For some applications, the anchoring location is located midway between the first bearing surface and the second bearing surface.

For some applications, the anchoring location is located closer to the first bearing surface than to the second bearing surface.

For some applications, the anchoring location is located closer to the second bearing surface than to the first bearing surface.

For some applications the wire defines: a first longitudinal segment between the head and the first bearing surface; a second longitudinal segment between the first bearing surface and the second bearing surface, and/or a third longitudinal segment between the second bearing surface and the terminus.

Within the steering region, the first longitudinal segment may be substantially parallel with the second longitudinal segment.

For some applications, within the steering region, the first longitudinal segment, the second longitudinal segment, and the third longitudinal segment are arranged in a force-multiplication arrangement.

For some applications, within the steering region, the second longitudinal segment is substantially parallel with the third longitudinal segment.

For some applications, the third longitudinal segment terminates at the terminus.

For some applications:

the steering region has a distal segment and a proximal segment,

the first and second longitudinal segments are present in the proximal segment and the distal segment, and/or

the third longitudinal segment is absent from the distal segment.

For some applications, the anchoring location is at a margin between the distal segment and the proximal segment.

For some applications, the wire is a first wire, the terminus is a first terminus, and the catheter further includes a second wire that extends from the head distally to a second terminus of the second wire, the second terminus being anchored to the tube.

For some applications:

the first wire is engaged with the tube in a manner in which tensioning of the first wire bends the steering region; and/or

the second wire is engaged with the tube in a manner in which tensioning of the second wire straightens the steering region.

For some applications, within the steering region:

the catheter includes a shaft within the circumferential wall,

the first wire is mounted medially with respect to the shaft, and/or

the second wire is mounted laterally with respect to the shaft.

For some applications, the second wire is arranged in a force-multiplication arrangement.

For some applications:

the force-multiplication arrangement is a second force-multiplication arrangement, the first wire being arranged in a first force-multiplication arrangement,

the first force-multiplication arrangement provides a first mechanical advantage, and/or

the second force-multiplication arrangement provides a second mechanical advantage that is different to the first mechanical advantage.

For some applications, within the steering region, the catheter includes a shaft within the circumferential wall, the shaft being configured to facilitate steering of the steering region.

For some applications, the shaft is substantially tubular.

For some applications, the shaft includes a chain of vertebrae mutually coupled at one side of the shaft and disjoined at another side of the shaft.

For some applications, the shaft includes a chain of vertebrae, at least one of the vertebrae defining:

a pair of curved protrusions, disposed bilaterally on opposite sides of the vertebra, and/or

a pair of curved cavities, disposed bilaterally on opposite sides of the vertebra.

For some applications, adjacent vertebrae of the chain are coupled to each other via mating between the pairs of curved protrusions and the pairs of curved cavities.

For some applications, the catheter includes a support ring that provides the bearing surface.

For some applications, the bearing surface is a static bearing surface over which the wire is slidable.

For some applications, the catheter includes a rotatable bearing that provides the bearing surface.

For some applications:

the tube extends from the proximal region to the steering region to define a longitudinal axis of the tube, and/or

the rotatable bearing is mounted in a manner that facilitates sliding of the wire around a transverse axis that is transverse to the longitudinal axis.

For some applications:

the tube extends from the proximal region to the steering region to define a longitudinal axis of the tube, and/or

the rotatable bearing is mounted in a manner that facilitates sliding of the wire around the longitudinal axis.

For some applications, the circumferential wall circumscribes, and thereby defines, a channel of the tube, and the rotatable bearing circumscribes the channel in a manner that facilitates sliding of the wire around the channel.

For some applications, the rotatable bearing includes a sheave.

For some applications, the apparatus further includes an extracorporeal interface.

For some applications, the extracorporeal interface is configured to receive the head of the catheter in a manner that operatively couples the extracorporeal interface to the steering region via the wire.

For some applications, the extracorporeal interface is configured to steer the steering region of the tube by applying a tensioning force to the first longitudinal segment.

There is further provided, in accordance with some applications, an assembly for facilitating an endoscopic procedure on a subject, the assembly for use with an extracorporeal interface, and including: a tube including: a proximal region; a steering region, distal from the proximal region, and dimensioned for advancement into the subject.

For some applications, the tube further includes a circumferential wall extending from the proximal region to the steering region; and/or at least one wire engaged with the tube.

For some applications, for each wire of the at least one wire:

the wire has a first longitudinal segment coupled to the extracorporeal interface at the proximal region and extending distally therefrom to a curved segment of the wire positioned at a curvature site arranged at the steering region;

the curved segment curves at the curvature site and connects the first longitudinal segment to a second longitudinal segment of the wire; and/or

the second longitudinal segment extends away from the curved segment, proximally along the steering region.

The wire may terminate at a terminus that is anchored to the tube at an anchoring location, the second longitudinal segment being positioned, along the wire, between the curved segment and the terminus.

For some applications, the steering region is a first steering region of the tube, and/or the tube further includes: at least one other steering region, disposed at a different axial location along the tube from the first steering region. For some applications, for each of the other steering regions, at least one respective other wire engaged with the tube.

For some applications, for each of the other wires:

the other wire has a first longitudinal segment coupled to the extracorporeal interface at the proximal region and extending distally therefrom to a curved segment of the other wire positioned at a curvature site arranged at the respective other steering region;

the curved segment curves at the curvature site and connects the first longitudinal segment to a second longitudinal segment of the other wire; and/or

the second longitudinal segment extends away from the curved segment, proximally along the respective other steering region.

The other wire may terminate at a respective terminus that is anchored to the tube at a respective anchoring location, the second longitudinal segment being positioned, along the other wire, between the curved segment and the terminus.

For some applications, the steering region is a distal region of the tube.

For some applications, at the steering region, the wire is arranged in a force-multiplication arrangement that, upon application of a tensioning force to a proximal end of the wire, multiplies the tensioning force within the steering region to apply a bending force that bends the steering region.

For some applications, the steering region is no more flexible than the proximal region.

For some applications, wherein the first longitudinal segment is substantially parallel with the second longitudinal segment.

For some applications, the tube extends from the proximal region to the steering region to define a longitudinal axis of the tube, and for each wire of the at least one wire, the wire is arranged such that, in response to tensioning of the wire, at the curvature site the wire slides around the longitudinal axis.

For some applications, the tube extends from the proximal region to the steering region to define a longitudinal axis of the tube, and for each wire of the at least one wire, the wire is arranged such that, in response to tensioning of the wire, at the curvature site the curved segment slides around a transverse axis that is transverse to the longitudinal axis.

For some applications, the tube is configured to be advanced through an anatomical lumen.

For some applications, the tube is configured to be advanced through an airway duct. For some applications, the second longitudinal segment terminates at the terminus.

For some applications, for at least one wire of the at least one wire:

the curvature site is a first curvature site,

the curved segment is a first curved segment, and/or

the second longitudinal segment extends proximally along the steering region to a second curved segment of the wire at a second curvature site.

For some applications, the wire further defines: a third longitudinal segment between the second curved segment and the terminus, and/or within the steering region, the third longitudinal segment is substantially parallel with the second longitudinal segment.

For some applications, within the steering region, the first longitudinal segment, the second longitudinal segment, and the third longitudinal segment are arranged in a force-multiplication arrangement.

For some applications, for at least one wire of the at least one wire, the anchoring location is located closer to the first curvature site than to the second curvature site.

For some applications, for at least one wire of the at least one wire, the anchoring location is located closer to the second curvature site than to the first curvature site.

For some applications, the anchoring location is located adjacent the first curvature site.

For some applications, the anchoring location is located adjacent the second curvature site.

For some applications, the anchoring location is located partway between the first curved segment and the second curved segment.

For some applications, the anchoring location is located midway between the first curved segment and the second curved segment.

For some applications, the at least one wire extends distally to the terminus.

For some applications, the circumferential wall circumscribes, and thereby defines, a channel of the tube.

For some applications, the channel is configured to facilitate passage of a tool therethrough.

For some applications, the channel is configured to facilitate passage of an imaging device therethrough.

For some applications, the curved segment at least partially circumscribes the channel at the curvature site.

For some applications, the second longitudinal segment extends to a second curved segment of the wire, the second curved segment at least partly circumscribing the channel.

For some applications, the curved segment circumscribes the channel in an arc of 330-360 degrees.

For some applications, the curved segment circumscribes the channel in an arc of 330 degrees or less.

For some applications, the curved segment circumscribes the channel in an arc of 180 degrees or less.

For some applications, the curved segment circumscribes the channel in an arc of 90 degrees or less.

For some applications, the at least one wire includes: a first wire, engaged with the tube in a manner in which tensioning of the first wire bends the steering region; and/or a second wire, engaged with the tube in a manner in which tensioning of the second wire straightens the steering region.

For some applications, the first wire includes a first-wire curved segment and the second wire includes a second-wire curved segment, the second-wire curved segment being disposed proximally from the first-wire curved segment.

For some applications, within the steering region, the tube includes a shaft within the circumferential wall, and/or the first wire is mounted medially with respect to the shaft, and/or the second wire is mounted laterally with respect to the shaft.

For some applications, the assembly further includes a third longitudinal segment of the wire which extends from the second curved segment and terminates at the terminus.

For some applications, the steering region includes a distal part and a proximal part; and/or the first and third longitudinal segments extend from the proximal part to the distal part.

For some applications, the second longitudinal segment extends from the distal part to the proximal part.

For some applications, the terminus is positioned at the distal part.

For some applications, the terminus is positioned at the proximal part.

For some applications, the terminus is positioned intermediate the distal part and the proximal part.

For some applications, within the steering region, the tube includes a shaft within the circumferential wall, the shaft being configured to facilitate steering of the steering region.

For some applications, the shaft is substantially tubular.

For some applications, the shaft includes a chain of vertebrae mutually coupled at one side of the shaft and disjoined at another side of the shaft.

For some applications, at least one of the vertebrae defines:

a pair of curved protrusions, disposed bilaterally on opposite sides of the vertebra, and/or

a pair of curved cavities, disposed bilaterally on opposite sides of the vertebra.

For some applications, adjacent vertebrae of the chain are coupled to each other via mating between the pairs of curved protrusions and the pairs of curved cavities.

For some applications, the tube includes a support ring on which the curved segment is mounted.

For some applications, the support ring defines a static bearing surface over which the curved segment is slidable.

For some applications, the tube includes a rotatable bearing mounted on the steering region and the wire is configured to move around the rotatable bearing.

For some applications, the tube extends from the proximal region to the steering region to define a longitudinal axis of the tube, and/or

the rotatable bearing is mounted in a manner that facilitates sliding of the wire around a transverse axis that is transverse to the longitudinal axis.

For some applications, the tube extends from the proximal region to the steering region to define a longitudinal axis of the tube, and/or the rotatable bearing is mounted in a manner that facilitates sliding of the wire around the longitudinal axis.

For some applications, the circumferential wall circumscribes, and thereby defines, a channel of the tube, and the rotatable bearing circumscribes the channel in a manner that facilitates sliding of the wire around the channel.

For some applications, the rotatable bearing includes a sheave.

For some applications, the assembly further includes the extracorporeal interface.

For some applications, the extracorporeal interface is configured to receive the proximal region of the tube in a manner that operatively couples the extracorporeal interface to the steering region via the at least one wire.

For some applications, the extracorporeal interface is configured to steer the steering region of the tube by applying a tensioning force to the first longitudinal segment.

For some applications, the first longitudinal segment, the curved segment, the second longitudinal segment, and terminus are arranged in a force-multiplication arrangement within the steering region.

There is further provided, in accordance with some applications, an assembly for facilitating a procedure on a subject, the assembly for use with an extracorporeal interface, and/or including a catheter that includes: a proximal region; and/or a steering region, distal to the proximal region, and dimensioned for advancement into the subject.

In some applications, the catheter may include a circumferential wall extending from the proximal region to the steering region. In some applications, the catheter may further include a wire, extending distally along the circumferential wall from the proximal region to the steering region.

For some applications, at the steering region, the assembly may be configured to define a force-multiplication arrangement that, upon application of a tensioning force to a proximal end of the wire, multiplies the tensioning force within the steering region to apply a bending force that bends the steering region.

For some applications, the force-multiplication arrangement is a first force-multiplication arrangement, and/or the wire is further arranged in a second force-multiplication arrangement that is configured to multiply the tensioning force by a different factor compared with the first force-multiplication arrangement.

For some applications, the wire has a first longitudinal segment coupled to the extracorporeal interface at the proximal region and extending distally therefrom to a curved segment of the wire positioned at a curvature site arranged at the steering region.

For some applications, the curved segment curves at the curvature site and connects the first longitudinal segment to a second longitudinal segment of the wire.

For some applications, the second longitudinal segment extends away from the curved segment, proximally along the steering region.

For some applications, the wire terminates at a terminus that is anchored to the catheter at an anchoring location, the second longitudinal segment being positioned, along the wire, between the curved segment and the terminus.

For some applications, the wire is arranged for force multiplication of the applied tensioning force by curving the curved segment at the curvature site, thereby subjecting each one of the first and second longitudinal segments with a tensioning force equivalent to the applied tensioning force.

For some applications, the steering region is a first steering region, and the at least one wire is at least one first wire.

For some applications, the catheter may include: a second steering region, proximal from the first steering region, and/or a second wire engaged with the catheter. In some applications, from the proximal end of the first wire, the first wire extends distally along the circumferential wall, past the first steering region to the second steering region, and/or at the second steering region, the assembly is configured to define a second force-multiplication arrangement that, upon application of a tensioning force to a proximal end of the second wire, multiplies the tensioning force within the second steering region to apply a bending force that bends the second steering region.

For some applications, the wire extends proximally to the terminus.

For some applications, the wire extends distally to the terminus.

For some applications, the force-multiplication arrangement is configured to define a distal segment of the steering region and a proximal segment of the steering region, and to multiply the tensioning force by a different factor in the distal segment compared with in the proximal segment.

For some applications, the wire is a first wire, and/or the assembly further includes a second wire.

For some applications, the second wire extends distally to a terminus that is anchored to the tube.

For some applications, the force-multiplication arrangement is a first force-multiplication arrangement, and/or the second wire is arranged in a second force-multiplication arrangement that is configured to multiply the tensioning force by a different factor in the distal segment compared with in the proximal segment.

There is further provided, in accordance with some applications, an assembly for facilitating an endoscopic procedure on a subject, the assembly for use with an extracorporeal interface, and including a tube that includes:

a proximal region;

a steering region, distal from the proximal region, and/or dimensioned for advancement into the subject; and/or

a circumferential wall circumscribing a longitudinal axis of the tube, and extending from the proximal region to the steering region along the longitudinal axis.

For some applications, the assembly includes a first wire, engaged with the tube, and/or defining a first-wire longitudinal segment extending, within the circumferential wall, along the steering region; and/or a second wire, engaged with the tube, and/or defining a second-wire longitudinal segment extending, within the circumferential wall, along the steering region.

For some applications, in the steering region, the second-wire longitudinal segment is disposed more laterally than is the first-wire longitudinal segment.

For some applications, the first-wire longitudinal segment is substantially parallel with the second-wire longitudinal segment.

For some applications, the first-wire and second-wire longitudinal segments are substantially parallel with the longitudinal axis.

For some applications, the first wire and the second wire are wires operable for steering the steering region for advancement of the tube into the subject.

For some applications, for each of the first wire and the second wire:

the wire has a first longitudinal segment coupled to the extracorporeal interface at the proximal region and extending distally therefrom to a curved segment of the wire positioned at a curvature site arranged at the steering region,

the curved segment curves at the curvature site and connects the first longitudinal segment to a second longitudinal segment of the wire, and/or

the second longitudinal segment extends away from the curved segment, proximally along the steering region, and terminates at a terminus that is anchored to the tube at an anchoring location, the second longitudinal segment being positioned, along the wire, between the curved segment and the terminus.

For some applications, the first wire is configured for bending at least the steering region and the second wire is configured for straightening at least the steering region.

For some applications, the first wire is configured for bending at least the steering region and is longitudinally arranged to extend at least partially along an internal surface of the circumferential wall; and/or the second wire is configured for straightening at least the steering region and is longitudinally arranged to extend at least partially along an external surface of the circumferential wall.

For some applications, the circumferential wall circumscribes, and thereby defines, a channel of the tube.

For some applications, the first-wire longitudinal segment and second-wire longitudinal segment are disposed at opposite sides of the channel.

For some applications, the steering region includes a tubular shaft within the circumferential wall, the tubular shaft formed with a circumferential shaft wall.

For some applications, the circumferential wall includes a flexible polymer and the tubular shaft is embedded within the flexible polymer.

For some applications, the first-wire longitudinal segment and second-wire longitudinal segment are disposed at opposite sides of the circumferential shaft wall.

For some applications, the circumferential shaft wall defines an internal surface and an external surface and first-wire longitudinal segment, and second-wire longitudinal segment are disposed at opposite sides of the circumferential shaft wall.

For some applications, tensioning of the first wire presses the first wire against the internal surface of the circumferential shaft wall and tensioning of the second wire presses the second wire against the external surface of the circumferential shaft wall.

There is further provided, in accordance with some applications, a method for use with an anatomical lumen of a subject, the method including:

advancing, towards the anatomical lumen, an assembly that includes:
a tube, having a proximal region, and a steering region distal from the proximal region, and/or
a wire, having a proximal end, extending along the tube to the steering region, and arranged within the steering region to define a force-multiplication arrangement; and/or

bending the steering region by applying a tensioning force to the proximal end of the wire such that the tensioning force is multiplied, within the steering region, by the force-multiplication arrangement.

For some applications, the method further includes multiplying the tensioning force by providing the wire with the force-multiplication arrangement at the steering region, thereby steering the assembly by use of a resultant multiplied tensioning force, and a magnitude of the tensioning force required for steering the tube equipped with the force-multiplication arrangement at the steering region is less than that required for steering a comparable tube that is unequipped with the force-multiplication arrangement.

For some applications:

the wire includes a first longitudinal segment extending to a curved segment of the wire positioned at a curvature site, the curved segment curving at the curvature site and connecting the first longitudinal segment to a second longitudinal segment of the wire, and/or

multiplying the tensioning force includes multiplying the tensioning force facilitated by at least one of the curved segment, the first longitudinal segment, and the second longitudinal segment.

For some applications, the assembly includes a bearing at the steering region, and bending the steering region includes bending the steering region by applying the tensioning force to the proximal end of the wire such that the wire slides around the bearing.

For some applications, the bearing is a rotatable bearing, and bending the steering region includes bending the steering region by applying the tensioning force to the proximal end of the wire such that the wire slides around the rotatable bearing.

The present invention will be more fully understood from the following detailed description of applications thereof, taken together with the drawings, in which:

### BRIEF DESCRIPTION OF THE DRA WINGS

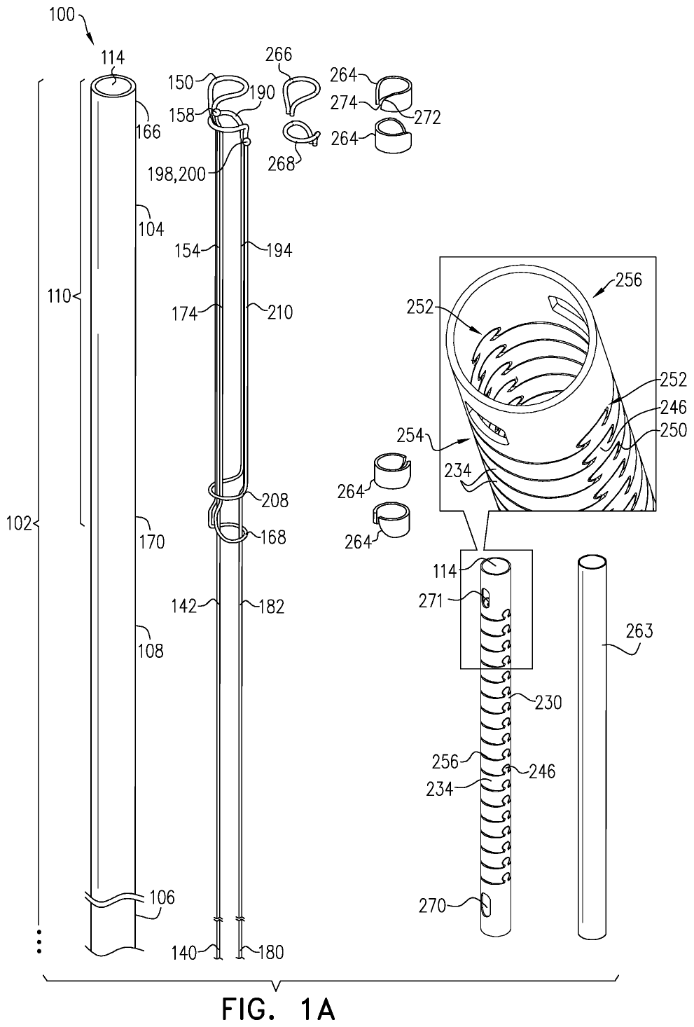

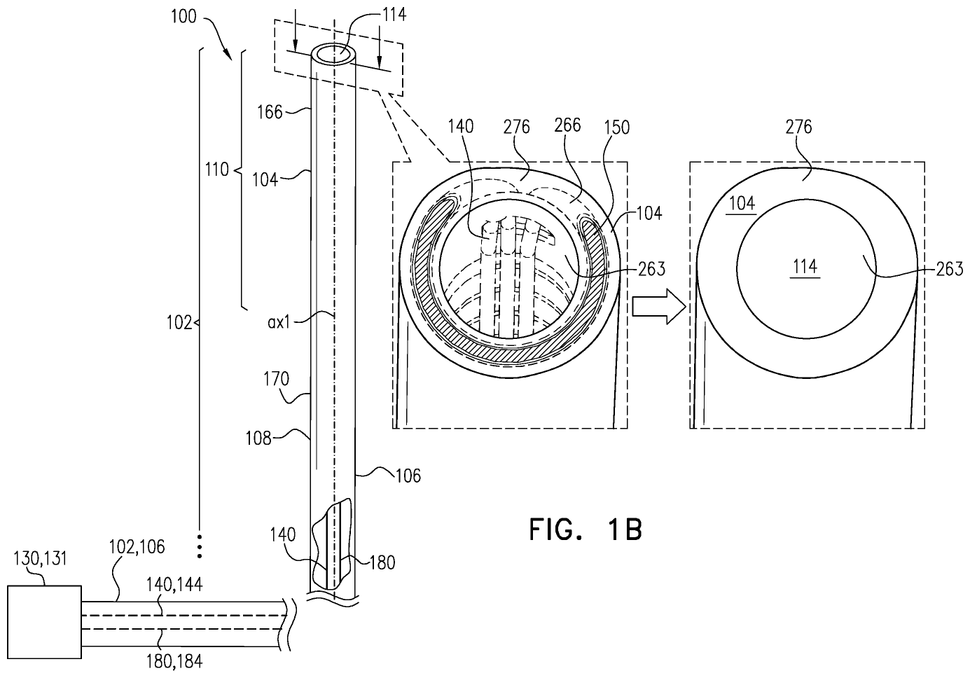

FIGS. 1A-1F are schematic illustrations of a tubular assembly for advancement into a subject during an endoscopic procedure, in accordance with some embodiments of the present disclosure;

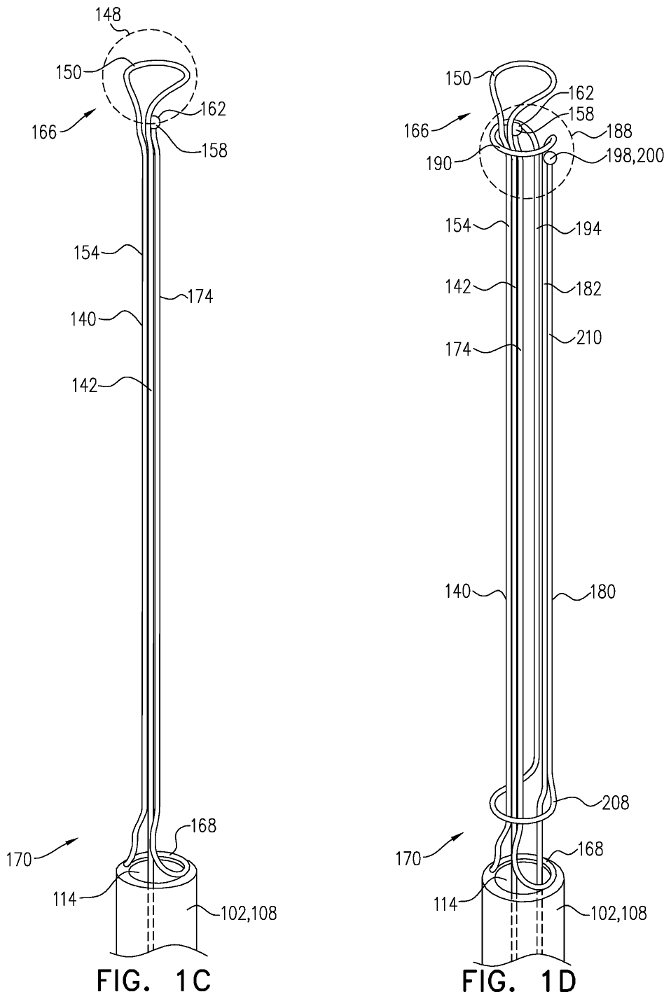

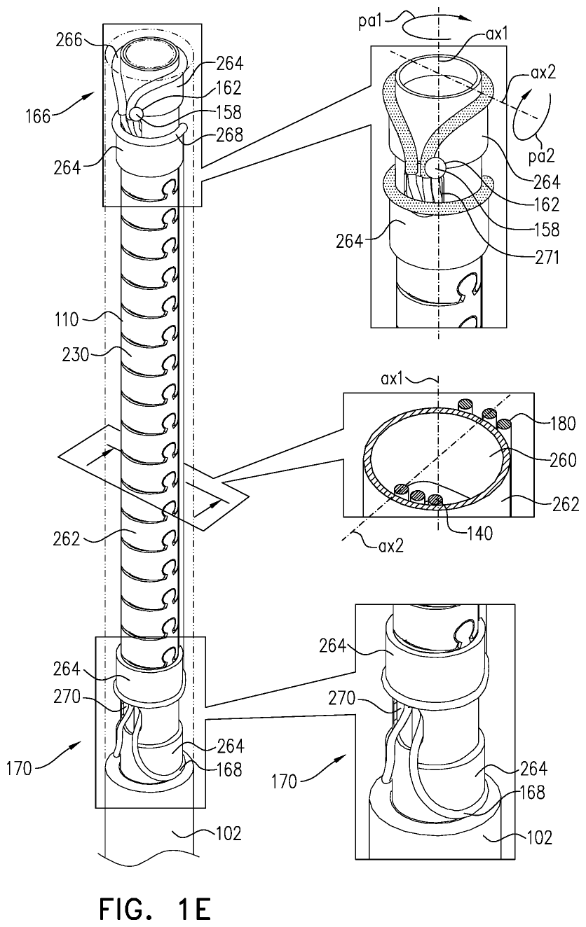

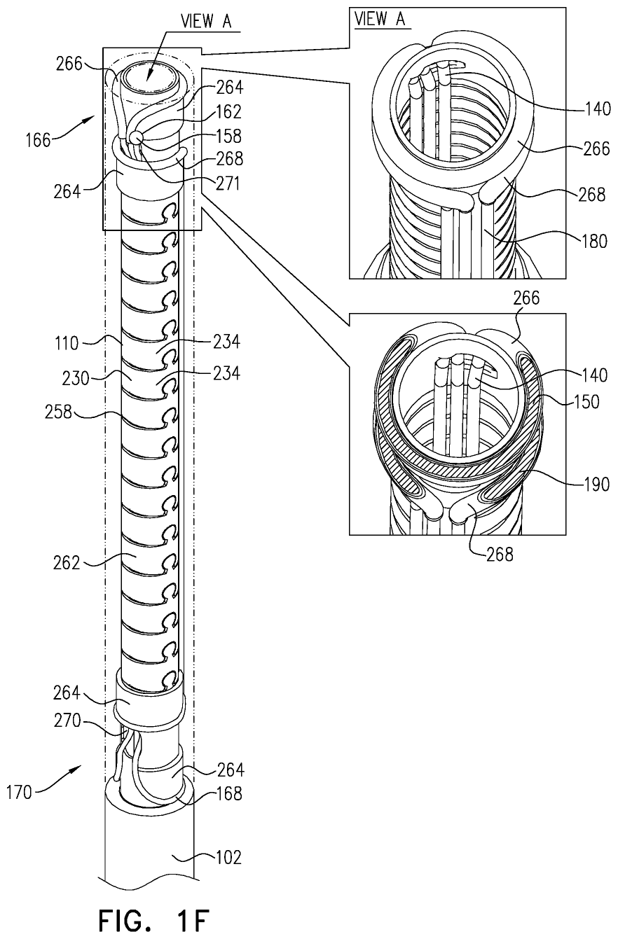

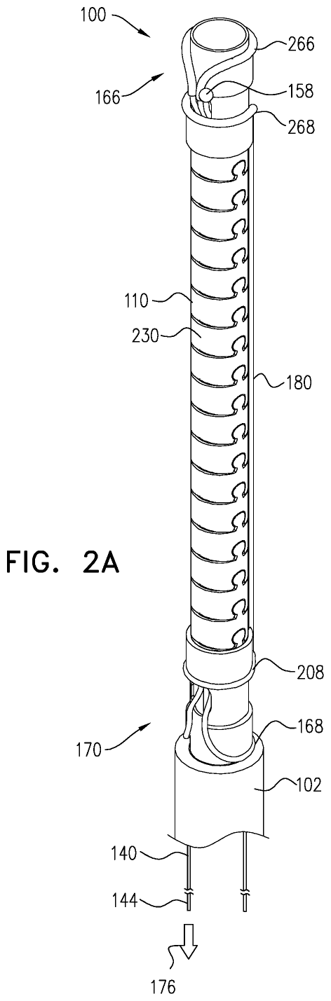

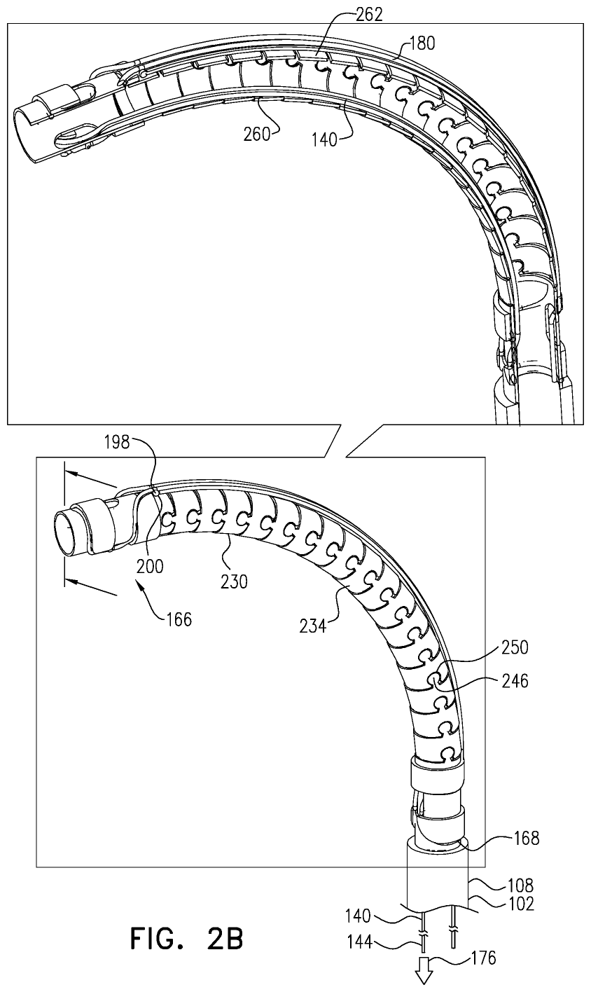

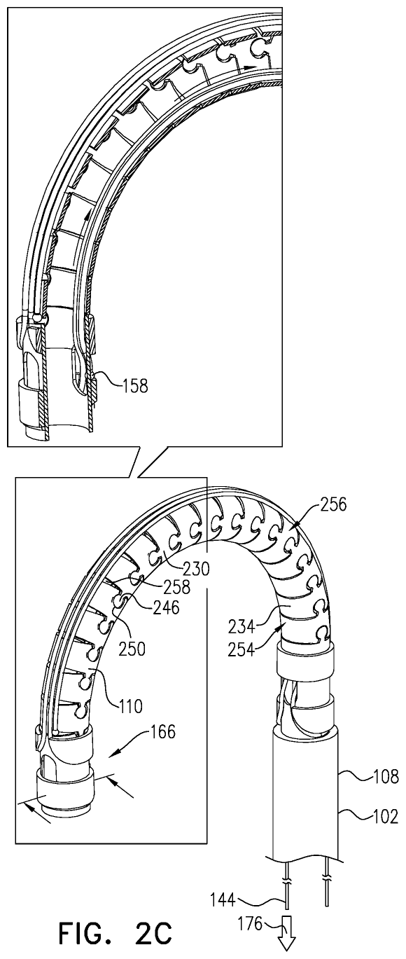

FIGS. 2A-2C are schematic illustrations of the tubular assembly at a bending operational mode, shown at an initial unbent (i.e., straight) stage, an intermediate partially bent stage and a fully bent stage, in accordance with some applications of the present disclosure;

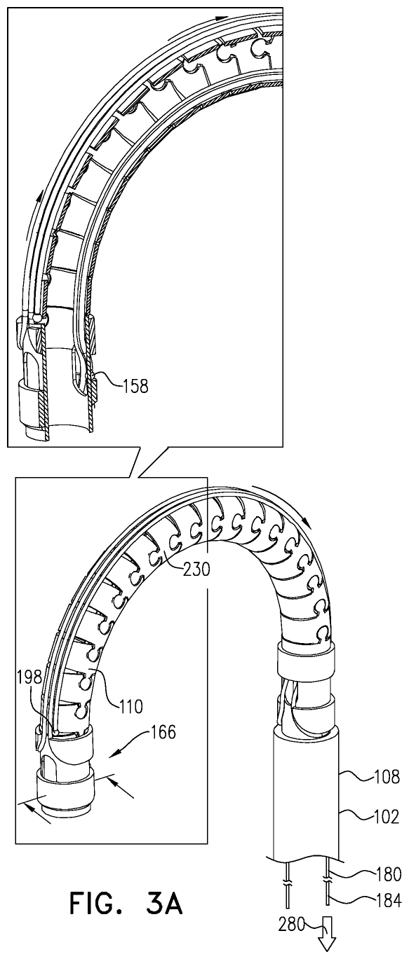

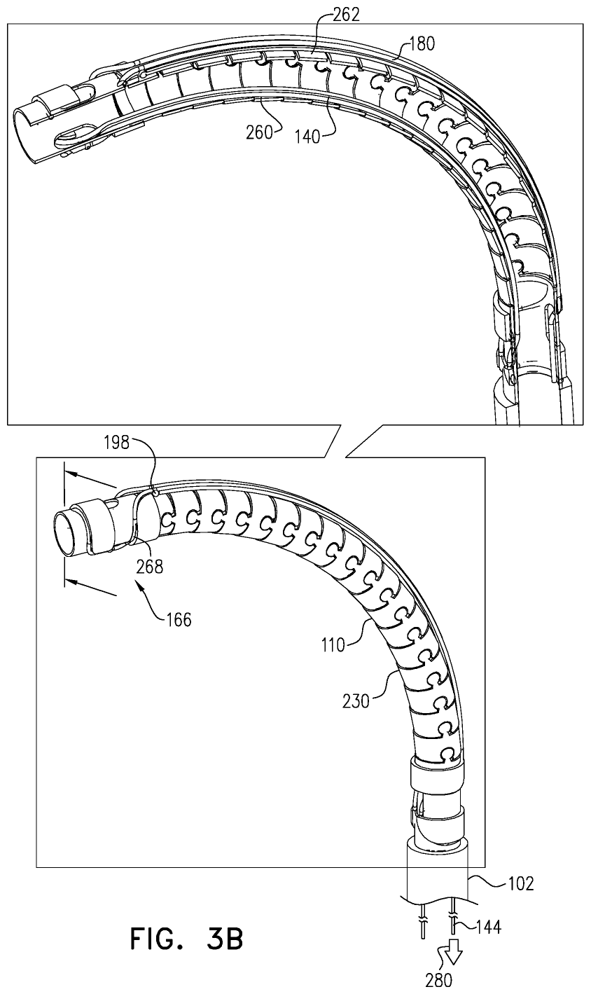

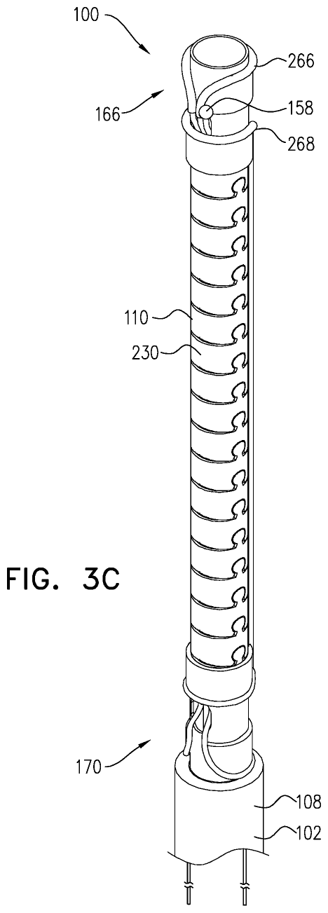

FIGS. 3A-3C are schematic illustrations of the tubular assembly at a straightening operational mode, shown at an initial bent stage, an intermediate partially straight stage and at a fully unbent (i.e., straight) stage, in accordance with some applications of the present disclosure;

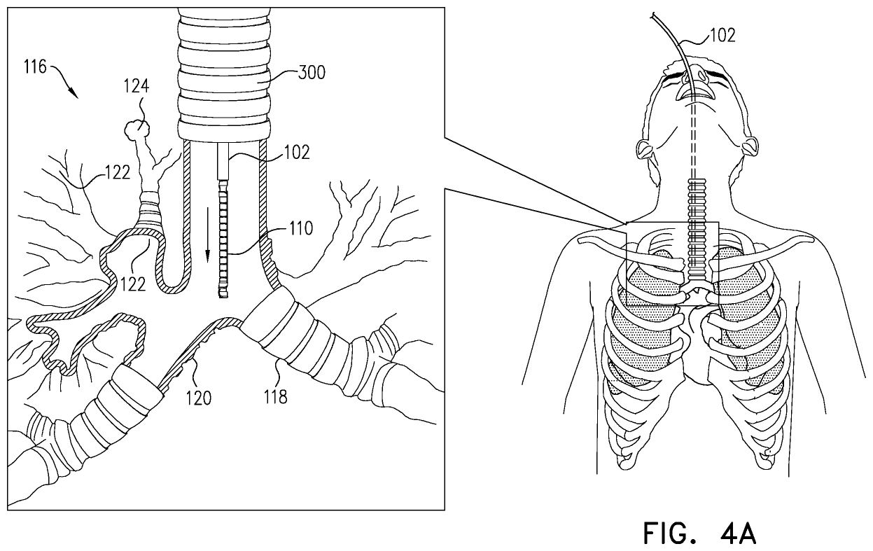

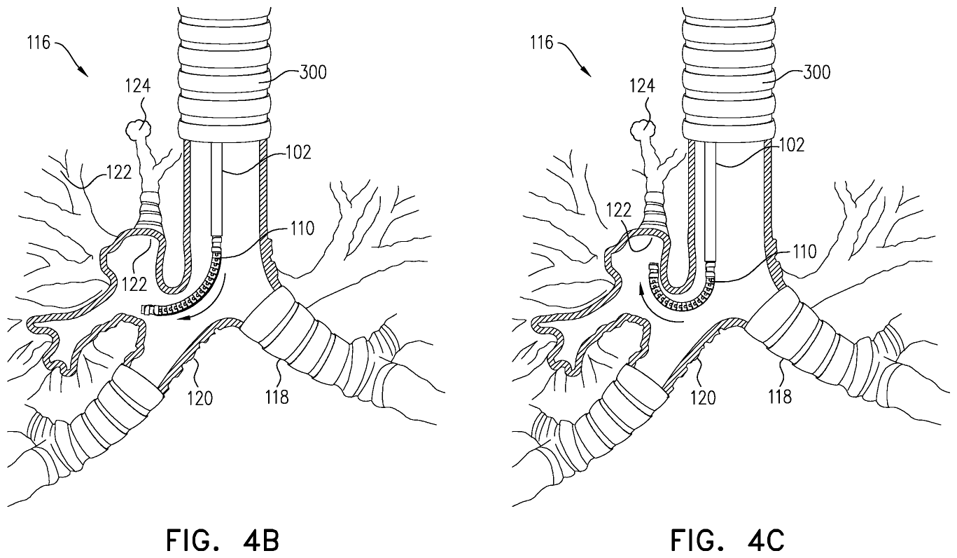

FIGS. 4A-4C are schematic illustrations of steering the tubular assembly within a human subject during an endoscopic procedure, shown at an initial straight stage, an intermediate partially bent stage, and a fully bent stage, in accordance with some applications of the present disclosure;

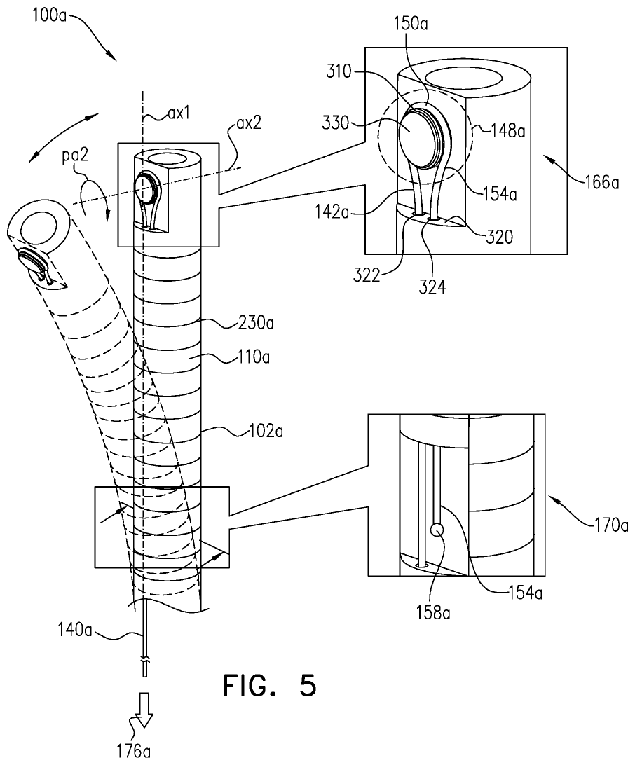

FIG. 5 is a schematic illustration of a tubular assembly, shown at a straight state and a bent state, in accordance with some applications of the present disclosure;

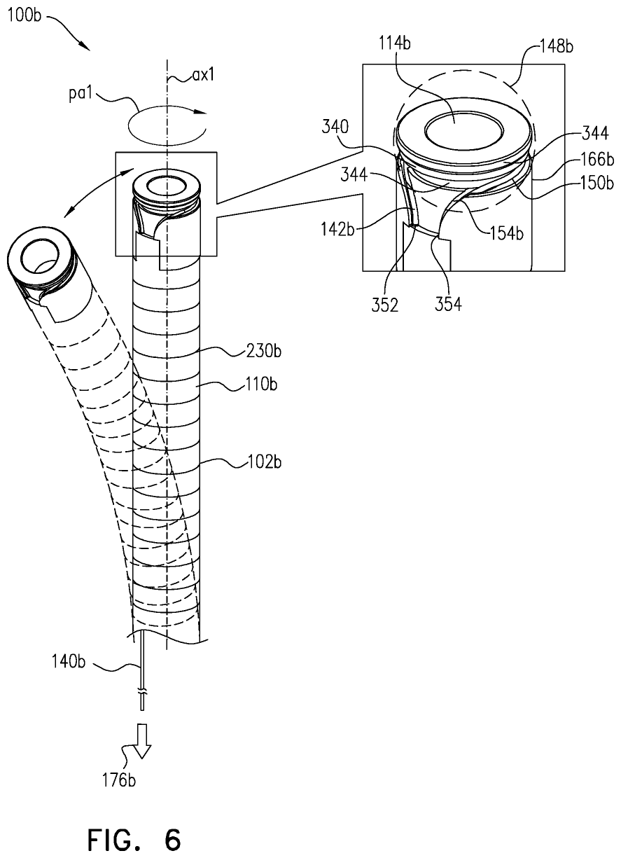

FIG. 6 is a schematic illustration of a tubular assembly, shown at a straight state and a bent state, in accordance with some applications of the present disclosure;

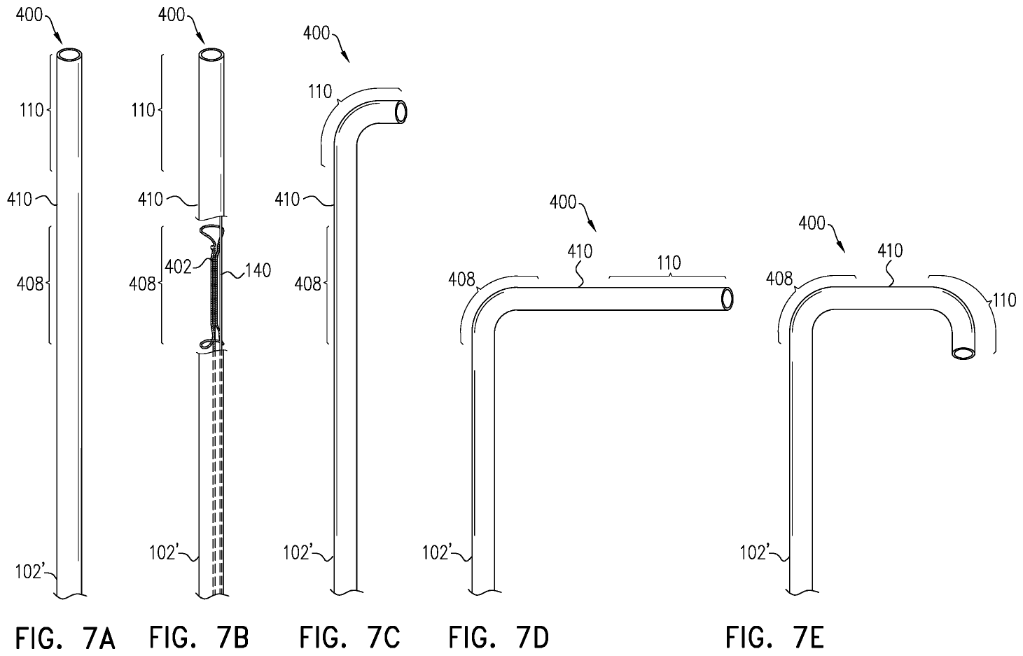

FIGS. 7A-7E are schematic illustrations of a tubular assembly, in accordance with some applications of the present disclosure; and

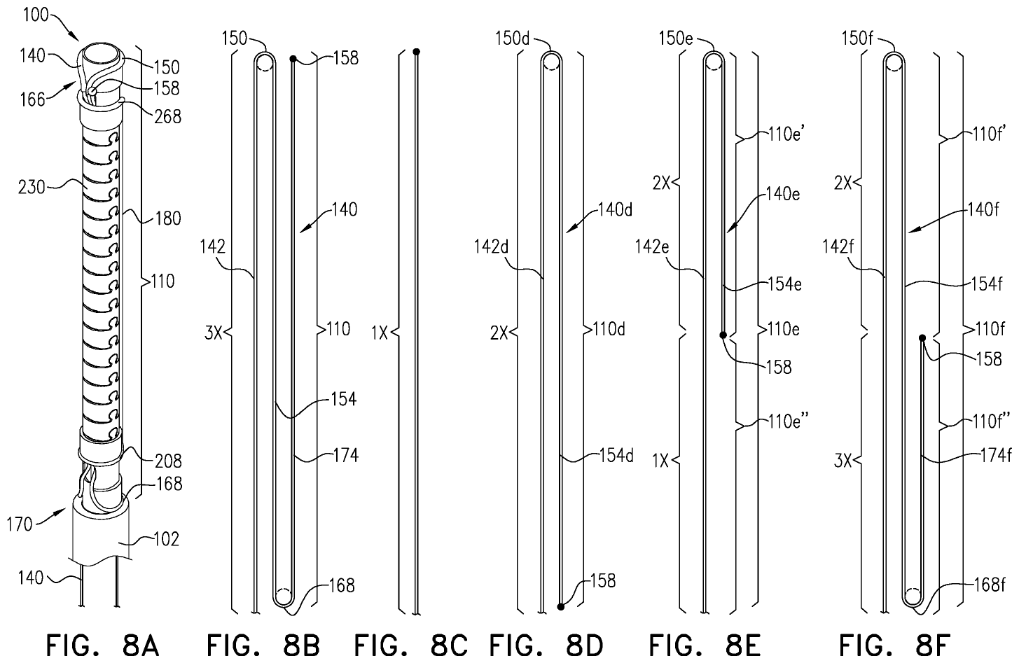

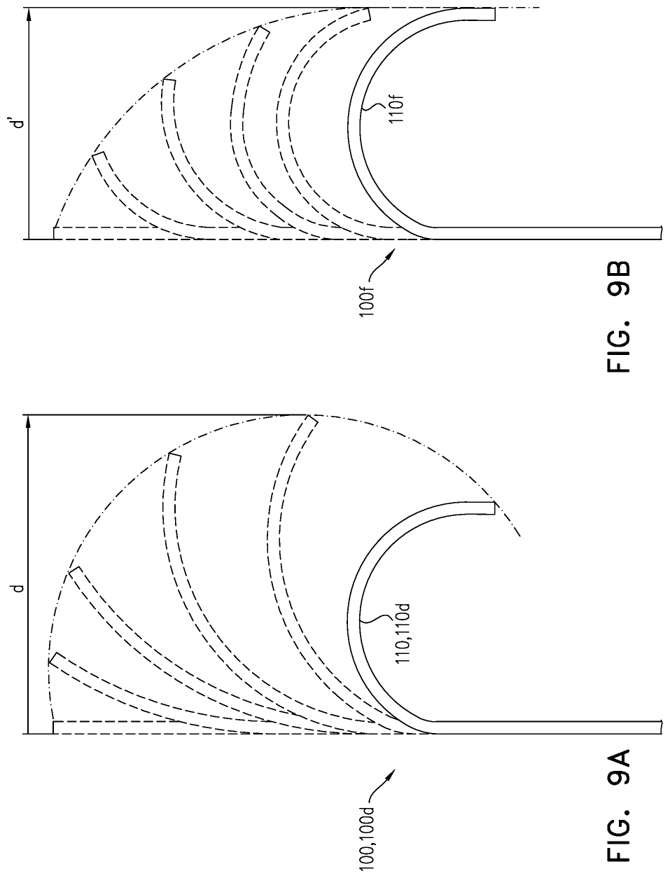

FIGS. 8A-8F and 9A-9B are schematic illustrations of a tubular assembly, in accordance with some applications of the present disclosure.

### DETAILED DESCRIPTION OF EMBODIMENTS

Reference is now made to FIGS. 1A-F, which are schematic illustrations of a tubular assembly 100 for advancement into a subject during an endoscopic procedure, in accordance with some embodiments of the present disclosure. FIGS. 1A-F show tubular assembly 100 in an unbent (e.g., straight) state thereof. FIG. 1A illustrates an exploded view of the tubular assembly, FIG. 1B illustrates the tubular assembly in an assembled state, FIG. 1C illustrates a single wire of the tubular assembly with some of the tubular assembly components omitted for clarity, FIG. 1D illustrates two wires of the tubular assembly with some of the tubular assembly components omitted for clarity, FIGS. 1E and 1F illustrate the tubular assembly with an upper portion of a tube removed for clarity.

Tubular assembly 100 comprises a tube 102 formed with a circumferential wall, such as a circumferential wall 104. Tube 102 may be an elongated tube and extends from a proximal region 106 of the tube, via an intermediate region 108 of the tube, to a distal steering region 110 of the tube, thereby defining a longitudinal axis ax1 of the tube. Tube 102 is facilitated for maneuvering (e.g., advancing and retracting) the tube 102 within an anatomical lumen, such as an airway duct.

For some applications, and as described in more detail hereinbelow, tube 102 comprises a shaft 230 at distal region 110. Typically, shaft 230 is substantially tubular.

Tubular assembly (e.g., tube 102 thereof) may be a catheter and is configured with a channel 114 (e.g., a lumen) defined by circumferential wall 104, which circumscribes the channel. Channel 114 typically serves as a channel (e.g., a working channel)—e.g., to receive an endoscopic tool or imaging device, such as for viewing and/or performing a procedure on a tissue of a subject. Such a procedure may include any form of endoscopy, such as in a non-limiting example, bronchoscopy wherein the catheter comprises a bronchoscope.

The lungs comprise a network of airway ducts forming the bronchial tree 116 (FIGS. 4A-C). The bronchial tree 116 includes the left main bronchus 118 and the right main bronchus 120, which each branch into a multifurcated network of airways including bronchi 122 and bronchioles, terminating at the alveoli 124. Advancing tube 102 through the airway ducts, and particularly via the network of tortuously branched bronchi 122 and/or bronchioles, is facilitated by steering of distal region 110. Steering the distal region 110 is actuated by an extracorporeal interface or controller 130 (FIG. 1B), which may be humanly, mechanically, manually, electronically, and/or automatically operated, or a combination thereof. Thus, extracorporeal interface 130 may be considered to comprise, to be a component of, or to control a steering manipulator.

For some applications, and as shown, extracorporeal interface 130 comprises or serves as a head 131 of tube (e.g., catheter) 102. For some applications, extracorporeal interface is configured to be functionally coupled to a head of tube 102—e.g., the tube has a head that is distinct from extracorporeal interface 130. For example, head 131 may be a component of tube 102. For some such applications, tube 102 may comprise a handle that comprises or serves as head 131. In any case, head 131 is coupled to proximal region 106 of tube (e.g., catheter) 102, and is operatively coupled to the steering region of the tube via one or more wires—e.g., as described herein.

It is to be noted that the term “steering” (including the specification and the claims) means active steering (as opposed to mere flexibility), involving bending and/or unbending (i.e., straightening), distal region 110. At least one wire 140 (e.g., a pullwire) is provided for facilitating the steering of distal region 110.

FIG. 1C shows a single wire 140 as it is positioned within tube 102, although for clarity, shaft 230 and distal region 110 of tube 102 are not shown in FIG. 1C. Further for clarity, FIG. 1C also does not show a second wire 180 that is typically also present within tube 102, and that is described in more detail hereinbelow. FIG. 1D shows both wire 140 and wire 180 as they are positioned within tube 102, although for clarity, shaft 230 and distal region 110 of tube 102 are not shown in FIG. 1D. Wire 140 has a first longitudinal segment 142, which extends distally along tube 102 (e.g. parallel with longitudinal axis ax1), from an actuation end 144 of the first longitudinal segment, engaged with extracorporeal interface 130 (FIG. 1B), to a curvature site 148 defined at distal region 110 (e.g. at a distal part (e.g. a distal limit) 166 of the distal region).

At curvature site 148, first longitudinal segment 142 reaches a curved segment 150 (e.g., a bight) of the wire, at which wire 140 curves to connect first longitudinal segment 142 to a second longitudinal segment 154 of the wire. Second longitudinal segment 154 extends from curved segment 150 and/or curvature site 148, proximally along tube 102. For some applications, second longitudinal segment 154 extends parallel with longitudinal axis ax1 (FIG. 1B) and/or alongside first longitudinal segment 142.

Curved segment 150 in FIGS. 1A-3C is arranged to at least partly circumscribe channel 114. In some applications, and as shown, curved segment 150 substantially completely circumscribes channel 114-i.e., forming a substantially complete 360-degree angle circle around the channel. In some applications, curved segment 150 circumscribes channel 114 in an arc of 360 degrees or less, e.g., an arc of 330 degrees or less, an arc of 330-360 degrees, an arc of 270 degrees or less, an arc of 180 degrees or less, an arc of 90 degrees or less, an arc of 45 degrees or less. In some applications, curved segment 150 is arranged to form a U-shaped loop, circumscribing channel 114 only minimally, as will be further described in detail in reference to FIG. 5.

Wire 140 terminates at a terminus 158 that is anchored, i.e., is fixedly coupled, to tube 102 at an anchoring location 162. Anchoring location 162 may be disposed at any location along tube 102 but is typically positioned within distal region 110. In the example shown, terminus 158 is disposed at distal part 166 of distal region 110.

Second longitudinal segment 154 extends proximally along distal steering region 110—e.g., to a proximal part (e.g., a proximal limit) 170 of distal steering region 110. In the example shown, second longitudinal segment 154 extends to a second curved segment 168 which at least partially circumscribes channel 114 at proximal part 170. In the example shown, a third longitudinal segment 174 of wire 140 extends from second curved segment 168, returning distally along tube 102—e.g., parallel with longitudinal axis ax1 (FIG. 1B) and/or alongside first and second longitudinal segments 142 and 154, respectively, and terminates at terminus 158.

It is to be noted that the term “wire” (including the specification and the claims) is not intended to be limited to a single strand, a particular cross-sectional shape, or a particular material. For example, wire 140 may be a solid wire, a stranded wire, a braided wire, etc. Wire 140 may comprise any suitable material such as a metal, a polymer, a ceramic, or a composite material.

Wire 140 is typically a bending wire, configured to bend at least a portion (e.g., the entirety) of distal region 110 upon application of a tensioning force (shown by an arrow 176 in FIG. 2A) to the wire at actuation end 144. Such tensioning of wire 140 slides first longitudinal segment 142 proximally with respect to tube 102, and due to the fixation of terminus 158 to the tube, the tensioning bends distal region 110, as seen in FIGS. 2A-C, and as described in more detail hereinbelow. Thus, tensioning force 176 may therefore be referred to as a bending force.

The arrangement of wire 140 described hereinabove may therefore be considered to be a force-multiplication arrangement (e.g., a pulley arrangement). Within this arrangement, each curved segment 150 serves as a force-multiplier by simulating the operation of a rope sliding around a sheave or wheel of a conventional movable pulley arrangement. The resultant multiplied force is distributed at curvature site 148, such that each of first longitudinal segment 142 and second longitudinal segment 154 is subjected to a tensioning force equivalent to tensioning force 176. Accordingly, forming wire 140 with one or more curved segments 150 facilitates steering of distal region 110 by multiplying the force of tensioning force 176. Thus, compared to a similar wire lacking curved segment(s), this arrangement may advantageously require significantly less tension to be applied to wire 140.

It is to be noted that the force-multiplication arrangement (and typically all force-multiplying features) of tubular assembly 100 may be contained within circumferential wall 104.

In conventional pulley systems the multiplied force increases in proportion to the number of ropes arranged to support a load and is referred to as the “mechanical advantage.” Accordingly in the present application, the multiplied force increases in proportion to the number of the longitudinal segments and/or curved segments supporting tube 102. In the example shown in FIG. 5 (described in more detail hereinbelow), wire 140 comprises a single curved segment 150 and first and second longitudinal segments 142 and 154, respectively. Accordingly, the degree of force multiplication is approximately twofold (ignoring mechanical losses, such as friction).

In some applications, wire 140 may comprise a plurality of curved segments and longitudinal segments, simulating a block and tackle system for increasing the degree of force multiplication. In a non-limiting example, as seen in FIG. 1C and further discussed herein below with respect to FIGS. 8A-B, wire 140 comprises first curved segment 150 and second curved segment 168 along with first, second and third longitudinal segments 142, 154, and 174, respectively. Accordingly, the degree of force multiplication is approximately threefold. Depending on the arrangement of the curved and longitudinal segments about tube 102, the mechanical advantage may be increased further, e.g., such that the degree of force multiplication is fourfold or more.

It is to be noted that wire 140 may comprise a single curved segment 150 (e.g., as shown for the embodiments of FIGS. 5 and 6), two curved segments (e.g., first curved segment 150 and second curved segment 168) or more. The same applies to wire 180, mutatis mutandis. The curved segments and terminus may be arranged at any location on tube 102. but as described herein, positioning them at distal region 110 of tube 102 contributes to the distal region acting as an active steering region—e.g., with middle region 108 and proximal region 106 of tube 102 remaining relatively passive. Furthermore, and as described in more detail herein below with respect to FIGS. 9A-B, concentrating the forces (e.g., the multiplied forces) and steering within distal region 110, reduces undesired and unintended curving of middle region 108 and proximal region 106 of tube 102, e.g., without requiring these regions to be modified to have increased rigidity. In some applications, the distal region 110 may comprise the same or less flexibility than the middle region 108 and/or than the proximal region 106.

It is typically advantageous for tube 102 to be narrow, so as to be advanceable through anatomical lumens such as the airways. For example, tube 102 may have an outer diameter of 1-16 mm, e.g., 1-12 mm (e.g., 1-10 mm, e.g., 2-9 mm, such as 2-5 mm or 4-9 mm), or 5-16 mm (e.g., 11-16 mm, 5-10 mm, or 7-14 mm, such as 8-12 mm). Tube 102 may have an inner diameter of 0.6-14 mm, e.g., 1-10 mm, e.g., 1-8 mm, e.g., 5-8 mm, or 1-5 mm, e.g., 1-4 mm (e.g. 1-3 mm, such as 1-2 mm) or 2-5 mm (e.g. 3-5 mm, such as 4-5 mm). For a pullwire steerable tube, such narrowness means that the pullwires will be close to the central longitudinal axis of the tube. This closeness results in the pullwires having less leverage to bend the tube, and therefore increased tensioning force is required. The inventors therefore suggest that the advantages of tube 102 and its force-multiplying wire arrangements are particularly pertinent for narrower tubes.

For applications in which terminus 158 is disposed within distal region 110, terminus 158 may be positioned at distal part 166 or proximal part 170 of the distal region, or alternatively partway between the distal part and the proximal part.

In some applications, distal region 110 may be configured to straighten automatically in response to release of tensioning force 176 applied upon wire 140—e.g., due to elasticity of the distal region. For some such applications, distal region 110 may comprise only a single wire (i.e., a bending wire). Thus, rather than FIG. 1C showing a single wire of a two-wire embodiment for the sake of clarity, as described hereinabove, FIG. 1C may in fact represent an embodiment in which distal region 110 comprises and is steered by a single wire.

In some applications, an additional wire—a straightening wire—is used to actively straighten distal region 110. For example, and as shown, a straightening wire 180 is provided in addition to bending wire 140 and straightens distal region 110 upon being tensioned at activation end 144—e.g., as seen in FIGS. 3A-C and as described in more detail hereinbelow. Similar to bending wire 140, straightening wire 180 has a first longitudinal segment 182, which extends distally along tube 102 (e.g., parallel with longitudinal axis ax1), from an actuation end 184 (FIG. 1B) of the first longitudinal segment, engaged with extracorporeal interface 130, to a curvature site 188 defined at distal region 110 (FIG. 1D). At curvature site 188, first longitudinal segment 182 reaches a curved segment 190 of the wire, which circumscribes channel 114 and connects first longitudinal segment 182 to a second longitudinal segment 194 of the wire. Second longitudinal segment 194 extends from curved segment 190 and/or curvature site 188, back proximally along tube 102. For some applications, second longitudinal segment 194 extends parallel with longitudinal axis ax1 (FIG. 1B) and/or alongside first longitudinal segment 182.

Straightening wire 180 terminates at a terminus 198 that is anchored, i.e., is fixedly coupled, to tube 102 at an anchoring location 200. Anchoring location 200 may be disposed at any location along tube 102. In some applications, anchoring location 200 is positioned within distal region 110. In the example shown, terminus 198 is disposed at distal part 166 of distal steering region 110. Second longitudinal segment 194 extends to a second curved segment 208 which circumscribes channel 114 at proximal part 170 of distal steering region 110. A third longitudinal segment 210 extends from the second curved segment 208 distally along tube 102 and parallel with longitudinal axis ax1 (FIG. 1B) and/or alongside first and second longitudinal segments 182 and 194 of wire 180, respectively, and terminates at terminus 198.

It is to be noted that straightening wire 180 may comprise a single curved segment 190, two curved segments (e.g., first curved segment 190 and second curved segment 208) or more—e.g., as described hereinabove with reference to bending wire 140, mutatis mutandis.

Bending wire 140 and straightening wire 180 extend proximally from distal region 110, and through middle region 108 and proximal region 106. As seen in FIGS. 1D-1F, for some applications, curved segment 190 of straightening wire 180 is disposed proximally from curved segment 150 of bending wire 140. Curved segment 190 may circumscribe longitudinal segments 142, 154, and 174 of bending wire 140. For some applications, and as shown, curved segment 208 of straightening wire 180 is disposed distally from curved segment 168 of bending wire 150. Curved segment 208 may circumscribe longitudinal segments 142, 154, and 174 of bending wire 140.

For some applications, and as shown, curved segments 150 and 168 of bending wire 140 are spaced further axially apart than are curved segments 190 and 208 of straightening wire 180.

For some applications, and as shown, longitudinal segments 142, 154, and 174 of bending wire 140 are disposed on an opposite side of longitudinal axis ax1 from longitudinal segments 182, 194, and 210 of straightening wire 180.

In some applications, within distal region 110 wires 140 and/or 180 are mounted within tube 102 itself (e.g., within channels defined in the tube). In some applications, the wires 140 and/or 180 are mounted on shaft 230, which itself is disposed within distal region 110 of tube 102. Shaft 230 serves as a conduit circumscribing channel 114. In some applications, shaft 230 comprises a hypotube-like conduit, e.g., formed with cuts, such as a mesh, a manifold or any other suitable structure which provides the shaft with sufficient strength to advance within the anatomical lumen, yet with sufficient flexibility allowing the shaft to bend within the tortuous anatomical lumens. In some applications the shaft 230 is substantially tubular.

In the example shown, shaft 230, which is configured as the hypotube-like conduit, is formed of a series of annular members, such as vertebrae 234 coupled to each other in a chain. For some applications, each vertebra defines pairs of curved protrusions 246 and cavities 250, bilaterally disposed at each of lateral sides 252 of the vertebrae (FIG. 1F). In some applications, vertebrae 234 are mutually coupled to each other by the pairs of curved (e.g., circular) protrusions 246, which are configured to mate with corresponding pairs of cavities 250 of an adjacent vertebra. Gaps 258 (FIG. 2C) between adjacent vertebrae 234 allow for flexible steering of shaft 230, with the gaps becoming narrower (e.g., closing entirely) at a first side 254 of the shaft while becoming wider at a second opposite side 256 of the shaft.

It is appreciated that shaft 230 may comprise any suitable configuration, such as a smooth tube, or any type of conduit.

For some applications, and as shown, longitudinal segments 142, 154, and 174 of bending wire 140 are mounted internally with respect to shaft 230 on an internal, circumferential wall 260 (FIG. 1E). For some applications, and as shown, longitudinal segments 182, 194, and 210 of straightening wire 180 are mounted externally with respect to shaft 230 at an external, circumferential wall 262 of the shaft. Thus, for some applications, and as shown, the longitudinal segments of bending wire 140 are disposed more medially (i.e., closer to axis ax1) than are the longitudinal segments of straightening wire 180. Namely, the longitudinal segments of straightening wire 180 are disposed more laterally (i.e., further from axis ax1) than are the longitudinal segments of bending wire 140.

Despite the different placement of bending wire 140 and straightening wire 180 with respect to shaft 230, this configuration results in each of these wires pressing against the shaft when the wire is tensioned. During bending of tube 102, tensioned wire 140 abuts internal, circumferential wall 260 of shaft 230, as seen in FIG. 2B, thereby pressing the wire against the internal wall. During straightening of tube 102, tensioned wire 180 abuts external, circumferential wall 262 of shaft 230, as seen in FIG. 3B, thereby pressing the wire against the external wall. Such an arrangement may advantageously improve the integrity of tubular assembly 100, e.g., by reducing a likelihood of a tensioned wire pulling away from its mounting. For example, if bending wire 140 were disposed at external wall 262 of shaft 230, upon bending of distal region 110 the tensioned bending wire might cut its way out of tube 102 (e.g., like a cheese wire) as it tries to take a shorter route between parts of the distal region. Thus, when the configuration shown is used, shaft 230 additionally serves as a reinforcement.

In some applications, and as shown, wire 140, wire 180, and/or shaft 230 are situated (e.g., embedded) within the material (e.g. material 276 (FIG. 1B), such as a polymer) of the tube 102 (i.e., within circumferential wall 104) at distal region 110. In some application, and as shown, a liner 263 (FIG. 1A) may be disposed within shaft 230, thereby serving as a smooth wall for channel 114/as a smooth inner surface of tube 102 (at least at distal region 110)—e.g., by partitioning the channel from the shaft and/or from wire 140. For some applications, liner 263 extends along the entirety of tube 102.

Support rings 264 (FIG. 1E) may be included within distal region 110, circumscribing channel 114. Curved segments 150, 168, 190 and 208 of wires 140 and 180 may be mounted on support rings 264 for low-friction sliding the curved segments over the support rings. Thus, support rings 264 may serve as static bearing surfaces for the curved segments of the wires. This is shown for support rings 264 on which curved segments 168 and 208 are mounted.

In some applications, the components of wires 140 and/or 180, disposed along external wall 262 of shaft 230, are situated within the material of the tube 102 at distal region 110. Thus, support rings 264 may provide mechanical reinforcement, while conduits 266 and 268 serve as static low-friction bearing surfaces for the curved segments of the wires. This is shown for the support rings on which curved segments 150 and 190 are mounted. In some applications, support rings 264 are formed in an annular configuration, e.g., entirely encircling channel 114. In some applications, support rings 264 only partially encircle channel 114 and/or taper to form a slot 274 at edges 272 for inserting the support rings on the shaft 230, as shown.

Any number of support rings 264 may be provided and are positioned at any suitable location along distal region 110. In some applications, as shown, each of curved segments 150, 168, 190 and 208 is mounted on a support ring 264, such that first and second support rings 264 are positioned at distal part 166 to support curved segments 150 and 190 of respective bending wire 140 and straightening wire 180 and third and fourth support rings 264 are positioned at proximal part 170 to support curved segments 168 and 208 of respective bending wire 140 and straightening wire 180.

In the example shown, second curved segment 168 of bending wire 140 circumscribes support ring 264 on external, circumferential wall 262, and the bending wire enters channel 114 of shaft 230 via an aperture 270 formed at a proximal part of the shaft. Bending wire 140 exits shaft 230, via an aperture 271 formed at a distal part of the shaft such that curved segment 150 can circumscribe support ring 264 on external, circumferential wall 262.

In some applications, where bending wire 140 or straightening wire 180 transitions from a curved segment to a longitudinal segment, the bending wire at least partially overlies the edges 272 of slot 274 formed in support rings 264 (see, for example, FIG. 1F).

In some applications, anchoring location 162 of terminus 158 of wire 140 and anchoring location 200 of terminus 198 of wire 180 are at distal part 166 of distal region 110. That is, in some applications, termini 158 and 198 are anchored to shaft 230 at distal part 166 of distal region 110 (FIGS. 1E and 2B).

It is to be noted that in some applications, shaft 230 and/or any one of support rings 264 may be omitted and wire 140 may be engaged with tube 102 in any suitable manner.

It is to be noted that in some applications, the arrangement of bending wire 140 and straightening wire 180 on tube 102 may be reversed. Furthermore, the function of wires 140 and 180 may be reversed such that wire 140 may operate as a straightening wire and wire 180 may operate as a bending wire.

Furthermore, although distal region 110 is shown as being steerable via two wires that are disposed opposite each other (i.e. circumferentially distributed at about 180 degrees around tube 102 from each other), for some applications the distal region of tubular assembly 100 or of other similar tubular assemblies may be steerable via more wires—e.g. three or four wires—to provide increased control over steering. For example, three wires may be circumferentially distributed at about 120 degrees around tube 102 from each other, or four wires may be circumferentially distributed at about 90 degrees around tube 102 from each other. Distal region 110 may therefore be considered to be a steering region. As explained hereinabove, this term indicates that it is actively steerable (as opposed to being merely flexible).

Although tube 102 is shown and described as having a single steering region (i.e., distal region 110), for some applications tube 102, or tubes of other similar tubular assemblies, may have multiple steering regions distributed along the tube at different axial locations. For such applications, each steering region of a given tube may be as described for distal region 110, mutatis mutandis. Furthermore, the steering regions of a given tube may be identical to each other (except for their axial position) or different from each other—e.g. with respect to the axial length of the steering region, the number of wires that are configured to steer the steering region, and/or the number of curved segments and longitudinal segments defined by each of the wires of the steering region. An example of a tubular assembly that comprises a tube having multiple steering regions is described hereinbelow with reference to FIGS. 7A-E.

Reference is now made to FIGS. 2A-C, which are schematic illustrations of tubular assembly 100 during a bending operational mode, shown at an initial straight stage (FIG. 2A), an intermediate partially bent stage (FIG. 2B), and a fully bent stage (FIG. 2C), in accordance with some applications of the present disclosure. A transition from the initial straight stage toward the partially and fully bent stages can be achieved by applying a tensioning force 176 to wire 140 (e.g., parallel to axis ax1) at actuation end 144, such as by using extracorporeal interface 130 (FIG. 1B). Due to the fixation of terminus 158 to tube 102, this tensioning bends distal region 110.

In the example shown in FIG. 2B, in the partially bent state distal part 166 of distal region 110 is bent, thereby becoming orthogonally angled with respect to middle region 108 of tube 102. In the example shown in FIG. 2C, in the fully bent state distal region 110 is substantially bent to a semi-circular arc such that distal part 166 is disposed at a substantially 180-degree angle with respect to middle region 108 of tube 102.

As described hereinabove, wire 140 comprises at least one curved segment 150 circumscribing channel 114 for providing tubular assembly 100 with the force-multiplication arrangement. By application of tensioning force 176 on wire 140, curved segment 150 operatively multiplies the tensioning force at curvature site 148 generating a resultant multiplied force. Accordingly, the steering of distal region 110 can be performed by application of a smaller degree of tensioning force 176, than would have been required in a tubular assembly which lacks the force-multiplication arrangement.

Reference is now made to FIGS. 3A-C, which are schematic illustrations of tubular assembly 100 during a straightening operational mode, shown at an initial bent stage (FIG. 3A), an intermediate partially straight stage (FIG. 3B) and at a fully straightened stage (FIG. 3C), in accordance with some applications of the present disclosure. A transition from the initial bent stage toward the partially and fully straightened stages can be achieved by applying a tensioning force 280 (which may therefore be referred to as a straightening force) to wire 180 (e.g., parallel to axis ax1) at actuation end 184, such as by using extracorporeal interface 130. Due to the fixation of terminus 198 to tube 102, this tensioning straightens distal region 110.

In the example shown in FIG. 3A, in the fully bent state distal region 110 is substantially bent to a semi-circular arc such that distal part 166 is disposed at a substantially 180° angle with respect to middle region 108 of tube 102. In the example shown in FIG. 3B, in the partially straightened state distal part 166 of distal region 110 is orthogonally angled with respect to middle region 108 of tube 102.

As described hereinabove, wire 180 comprises at least one curved segment 190 circumscribing channel 114 for providing tubular assembly 100 with the force-multiplication arrangement. By application of tensioning force 280 on wire 180, curved segment 190 operatively multiplies the tensioning force at curvature site 188 generating the resultant multiplied force. Accordingly, the steering of distal region 110 can be performed by application of a smaller tensioning force 280, than would have been required in a tubular assembly which lacks the force-multiplication arrangement.

It is to be noted that the examples of a “partially bent stage” and a “fully bent stage” are intended to be purely illustrative, and not limiting. For example, the “partially bent stage” is typically not a discrete stage. Similarly, the angle of distal part 166 with respect to middle region 108 may be different to that shown in the figures.

Reference is now made to FIGS. 4A-C, which are schematic illustrations of steering tubular assembly 100 within a human subject during bronchoscopy, in accordance with some applications of the present disclosure. FIG. 4A shows tube 102 being advanced into trachea 300 via an orifice (e.g., a mouth or a nose) of the subject—e.g., while in an initial straightened state. FIGS. 4B-C show further advancement of tube 102 into the bronchial tree at right main bronchus 120, e.g., facilitated by partial bending of the tube. A site (e.g., the airway 122) to which it is desired that tube 102 is advanced is shown to be along a bronchus that is disposed at an acute angle with respect to the trachea. Advancement of tube 102 around such angles, and generally through the tortuously branched bronchi of the lung is facilitated by steering of distal region 110. The structure of tubular assembly 100 may advantageously facilitate controlled and precise steering at such large angles. For example, the force-multiplication arrangement facilitates steering of tube 102 to a required position by applying a relatively low magnitude of tensioning force 176 and/or 280 in comparison with a magnitude of tension that would have been required for steering a similar tube that lacks the force-multiplication arrangement.

In some applications, curved segment 150 circumscribes channel 114, e.g., along a curved path pa1, which curves around longitudinal axis ax1 (FIG. 1E). In some applications, curved segment 150 may curve around tube 102 at other orientations, e.g., to circumscribe a transverse axis (e.g., a polar axis) ax2 along a path pa2, which curves around axis ax2, as will be further described in reference to FIG. 5.

Reference is now made to FIG. 5, which is a schematic illustration of a tubular assembly 100a, which is a variant of tubular assembly 100, in accordance with some applications of the present disclosure. For some applications, tubular assembly 100a is as described for tubular assembly 100 except where noted. Suffix “a” represents a corresponding element described in reference to tubular assembly 100.

Whereas tubular assembly 100 is configured such that the curved segments of its wires slide over static bearing surfaces, tubular assembly 100a is configured such that the curved segments of its wires move around a rotatable bearing such as a pulley wheel 310 (e.g., a sheave). Although a particular embodiment of tubular assembly 100a is shown (e.g., comprising a single wire that has a single curved segment), it is to be noted that the description of tubular assembly 100a is intended to teach more generally that, for some applications, rotatable bearings may be used in tubular assemblies such as tubular assembly 100, mutatis mutandis.

Furthermore, the description of tubular assembly 100a teaches that the second longitudinal segment 154a of wire 140a may be coupled to terminus 158a, such that further segments, such as third longitudinal segment 174 (FIG. 1C), are obviated.

Moreover, the description of tubular assembly 100a teaches that, for some applications, terminus 158a may be disposed at locations other than distal part 166a of distal steering region 110a, such as at proximal part 170a.

Moreover, the description of tubular assembly 100a teaches that, for some applications, wire 140a may comprise a single curved segment 150a, while the second curved segment 168 (FIG. 1C) is obviated.

In the example shown in FIG. 5, curved segment 150a is configured in a U-like shaped loop, at least partially encircling a pulley wheel 310. Curved segment 150a, mounted on pulley wheel 310, extends to second longitudinal segment 154a of the wire. A recess 320 for mounting the pulley wheel 310 therein may be formed along distal steering region 110a at any suitable location, such as at distal part 166a.

In the example shown in FIG. 5, first longitudinal segment 142a protrudes from shaft 230a or any portion of distal steering region 110a from a first bore 322 to pulley wheel 310. Second longitudinal segment 154a penetrates shaft 230a via a second bore 324. Pulley wheel 310 may be fixed to steering region 110a at recess 320 via a mounting element 330, such as an axle.

Tensioning of wire 140a causes first longitudinal segment 142a to slide proximally with respect to tube 102a and around pulley wheel 310. Wire 140a moves about axis ax2 along path pa2. Due to the fixation of terminus 158a to the tube, the tensioning force bends steering region 110a, e.g., as described hereinabove, mutatis mutandis. Curved segment 150a, revolving around pulley wheel 310, operatively multiplies a tensioning force 176a applied to wire 140a, e.g., as described for curved segment 150, mutatis mutandis.

Reference is now made to FIG. 6, which is a schematic illustration of a tubular assembly 100b, which is a variant of tubular assembly 100, in accordance with some applications of the present disclosure. For some applications, tubular assembly 100b is as described for tubular assembly 100 except where noted. Suffix “b” represents a corresponding element described in reference to tubular assembly 100.

Whereas tubular assembly 100 is configured such that the curved segments of its wires slide over static bearing surfaces, tubular assembly 100b is configured such that the curved segments of its wires move around a rotatable bearing such as a pulley wheel (e.g., a sheave). In this regard, tubular assembly 110b is similar to tubular assembly 110a.

However, in the embodiment of tubular assembly 100a the wire is configured to move around the rotatable bearing about transverse axis ax2 along path pa2, while in the embodiment of tubular assembly 100b the wire is configured to move around the rotatable bearing about longitudinal axis ax1 along path pa1.

In the example shown in FIG. 6, a rotatable bearing such as a pulley wheel 340 or any other annular shaped element is disposed at distal part 166b of distal steering region 110b. Pulley wheel 340 may be disposed at a peripheral recess 344 that circumscribes channel 114b.

Curved segment 150b, mounted on pulley wheel 340, extends to second longitudinal segment 154b of the wire. In the example shown in FIG. 6, first longitudinal segment 142b protrudes from shaft 230b or any portion of steering region 110b from a first bore 352 to pulley wheel 340 and second longitudinal segment 154b penetrates shaft 230b via a second bore 354.

Tensioning of wire 140b causes first longitudinal segment 142b to slide proximally with respect to tube 102b and around pulley wheel 340. Wire 140b at least partially moves about axis ax1 along path pa1. Curved segment 150b, revolving around pulley wheel 340, operatively multiplies a tensioning force 176b applied to wire 140b, e.g., as described for curved segment 150, mutatis mutandis.

Reference is now made to FIGS. 7A-E, which are schematic illustrations of a tubular assembly 400, which is a variant of tubular assembly 100 in which tube 102 has multiple (e.g., two, three, four, or more) steering regions distributed along the tube at different axial locations, in accordance with some applications. Tubular assembly 400 comprises a tube 102′, which may be identical to tube 102 of tubular assembly 100, except to accommodate the differences noted.

FIGS. 7A-B shows tube 102 in a straight state, with FIG. 7B including a cutaway to expose wires within the tube. Tube 102′ includes distal steering region 110, which for tubular assembly 400 defines a first steering region (e.g., a distal steering region). Additionally, tube 102′ has a second steering region 408, disposed at a different axial position to region 110. In the example shown, region 408 is proximal from steering region 110 (e.g., region 408 is a proximal steering region). Furthermore, in the example shown, a passive region 410 (e.g., a non-steering region, which may nonetheless be flexible) is defined axially between steering regions 110 and 408.

For simplicity, each of steerable regions 110 and 408 is shown as having, and being steerable via, a single pullwire (e.g., as for FIG. 1C, mutatis mutandis), but it is to be understood that each steerable region could have two, three, four, or more pullwires—e.g., as described hereinabove for tubular assembly 100, mutatis mutandis.

In FIG. 7B, a wire (i.e., a pullwire) 402 extends, from the proximal portion of tube 102′, to steerable region 408, where the wire is arranged into a force-multiplication arrangement having multiple longitudinal segments separated by one or more curved segments, e.g., as described hereinabove for wire 140 and steerable region 110, mutatis mutandis. Wire 402 may be similar or identical to wire 140 or wire 180. Steerable region 408 may comprise a shaft that is similar or identical to shaft 230, although for clarity this shaft is not shown in the cutaway of FIG. 7B. As discussed in more detail hereinbelow, any wires that are configured to bend steering region 110 (in this case wire 140) extend through steerable region 408.

FIG. 7C shows tubular assembly 400 during bending of steering region 110 without bending of steering region 408, by tensioning wire 140 without tensioning wire 402. FIG. 7D shows tubular assembly 400 during bending of steering region 408 without bending of steering region 110, by tensioning wire 402 without tensioning wire 140. FIG. 7E shows tubular assembly 400 during bending of both steering regions as both wires are tensioned. FIGS. 7C-E are not intended to indicate a limited number of discrete bending states of tubular assembly 400, but rather to illustrate that bending regions 110 and 408 are substantially independent of each other.

This independence between bending regions is provided at least in part by the force-multiplication arrangement at each bending region. For example, were a simple (e.g., linear) pullwire to be used to bend steering region 110, any force applied by the pullwire to region 110 upon tensioning of the pullwire would also be applied (e.g., substantially equally) to steering region 408. Thus, if steering region 408 were as flexible as steering region 110, tensioning the pullwire that is intended to bend region 110 would likely also bend region 408. However, for tubular assembly 400, the force-multiplication arrangement disclosed herein significantly reduces the magnitude of tensioning force required to be applied to wire 140 in order to bend steering region 110. Tube 102′ may be configured such that this reduced force is insufficient to bend steering region 110 or steering region 408 in the absence of such force-multiplication. Because wire 140 is arranged in a force-multiplication arrangement within steering region 110 but not within steering region 408, tensioning of wire 140 may exert sufficient force on steering region 110 to bend steering region 110 without exerting sufficient force on steering region 408 to bend steering region 408. Thus, for example, the bending state shown in FIG. 7C is possible without requiring bending region 408 or passive region 410 to be less flexible than bending region 110.

Steering of tubular assembly 400 through tortuous anatomical lumens, such as airways, may be advantageously enhanced by its ability to be bent multiple discrete steering regions.

Steering regions 110 and 408 of tube 102′ may be identical to each other (except for their axial position). However, the steering regions may alternatively be different from each other. For example, the steering regions may have different axial lengths from each other, may have a different number of pullwires, and/or a different mechanical advantage due to each of their wires having a different number of curved segments and longitudinal segments compared to the other steering region(s). Furthermore, the circumferential orientation of the pullwires (e.g., their longitudinal segments and/or their curved segments) with respect to the circumference of tube 102′ may be different for one steering region compared to another steering region. In the particular example shown, wires 140 (which bends region 110) and 402 (which bends region 408) are disposed in approximately the same circumferential orientation, and hence when tensioned they both bend their respective steering region to the right (from the perspective of the viewer).

Reference is now made to FIGS. 8A-F, which are schematic illustrations of various tubular assemblies, in accordance with some applications.

FIG. 8A shows tubular assembly 100 which, as described hereinabove, comprises, inter alia, tube 102, wire 140, and wire 180. FIG. 8B shows the arrangement of wire 140 but using a simplified schema in order to illustrate the pulley arrangement of the wire within the tubular assembly. FIG. 8C is provided purely for comparison, using the same simplified schema to show the arrangement of a single pullwire as may be found in an existing prior art catheter.

Returning to FIGS. 8B, wire 140 has two curved segments 150 and 168 and three longitudinal segments 142, 154, and 174. Wire 140 terminates at terminus 158, which is anchored to the tube 102, thereby defining an anchoring location. In the example shown, terminus 158 (and thereby the anchoring location) is positioned at the distal end of steering region 110. Thus, each of longitudinal segments 142, 154, and 174 extends along the entire length of steering region 110, and the pulley arrangement of wire 140 provides substantially consistent mechanical advantage throughout the length of the steering region.

FIGS. 8D, 8E, and 8F show, using the same simplified schema, wires 140d, 140c, and 140f, which may be considered to be variants of wire 140. In each case, the wire has a pulley arrangement in which multiple longitudinal segments of the wire are separated by curved segments of the wire, thereby providing force-multiplication properties, as described in general elsewhere herein.

Wire 140 (FIG. 8B) has three longitudinal segments 142, 154, and 174, and therefore provides a force multiplication of 3× (e.g., relative to that of the wire shown in FIG. 8C). As noted hereinabove, wire 140 provides such force multiplication substantially consistently along the entire length of steering region 110. Wire 140d (FIG. 8D) has two longitudinal segments 142d and 154d and a single curved segment 150d therebetween, thereby providing a force multiplication of 2× (e.g., relative to that of the wire shown in FIG. 8C). Again, this force multiplication is substantially consistent along the entire length of its corresponding steering region 110d.

In some applications, the wire may be arranged to provide a distal segment of the steering region with a different mechanical advantage to a proximal segment of the steering region. For example, the terminus of the wire may be disposed (e.g., anchored) partway along steering region 110, such that a segment of the steering region that is distal to the terminus has a mechanical advantage that is different from that of a segment of the steering region that is proximal from the terminus. For example, wire 140c (FIG. 8E) provides a distal segment 110c′ of steering region 110e with a mechanical advantage that is greater than that of a proximal segment 110e″ of the steering region, whereas wire 140f (FIG. 8F) provides a proximal segment 110f″ of steering region 110f with a mechanical advantage that is greater than that of a distal segment 110f of the steering region.

Similarly, to wire 140d, wire 140e (FIG. 8E) has two longitudinal segments 142e and 154c and a single curved segment 150e therebetween. However, terminus 158 of wire 140c is disposed partway (e.g., midway) along steering region 110e, such that longitudinal segment 154c is disposed within a distal portion of the steering region but not in a proximal portion of the steering region. That is, longitudinal segment 154e extends proximally from curved segment 150e, terminating partway along steering region 110e. Thus, the pulley configuration of wire 140e provides the distal portion of steering region 110e with a force multiplication of 2×, whereas the proximal half of the steering region is not provided with force multiplication.

Similarly, to wire 140, wire 140f (FIG. 8F) has three longitudinal segments 142f, 154f, and 174f, with curved segment 150c between longitudinal segments 142f and 154f, and curved segment 168c between longitudinal segments 154f and 174f. In contrast to wire 140, however, terminus 158 of wire 140f is disposed partway (e.g., midway) along steering region 110f, such that longitudinal segment 174f is disposed within a proximal portion of the steering region but not in a distal portion of the steering region. That is, longitudinal segment 174f extends distally from curved segment 168f, terminating partway along steering region 110f. Thus, the pulley configuration of wire 140f provides the distal portion of steering region 110f with a force multiplication of 2×, whereas the proximal portion of the steering region is provided with a force multiplication of 3×.

Further implications of the differences in mechanical advantage on bending of steering region 110 are shown in FIGS. 9A-B.

FIGS. 9A-B show schematic representations of tube assemblies (e.g., steering regions thereof) in various states of bending, according to some applications. In each of FIGS. 9A-B, solid lines represent the steering region in a bent state (e.g., fully bent), while broken lines represent the steering region in various less-bent states. Thus, each figure illustrates a bending curve that the steering region may follow on its way to the bent state. FIG. 9A represents a bending curve that may be followed by the steering region of a tubular assembly in which a wire is arranged to provide force multiplication that is substantially consistent along the entire steering region—e.g. steering region 110 or steering region 110d. FIG. 9A may therefore be considered to represent a distal part of tubular assembly 100 (that has steering region 110) or a tubular assembly 100d (that has steering region 110d). FIG. 9B represents a bending curve that may be followed by the steering region of a similar tubular assembly in which a wire is arranged to provide force multiplication that is greater at a proximal portion of the steering region compared with at a distal portion of the steering region—e.g. steering region 110f, which may be the steering region of a tubular assembly 100f.

In general, the radius of curvature of the steering region in FIG. 9B is smaller, throughout its bending toward its bent state, compared with that of FIG. 9A. Such a smaller radius of curvature may advantageously facilitate navigation of narrow airways and/or acute angles of turning. For example, a maximal lateral extent of the steering region during its bending (i.e. the maximum distance by which the distal end of the steering region extends laterally—e.g. from the proximal end of the steering region and/or from an axis of the tubular assembly just proximally from the steering region) may be reduced by providing greater force multiplication within the proximal portion of the steering region. Compare, for example, maximal lateral extent d in FIG. 9A with smaller maximal lateral extent d′ in FIG. 9B.

The effect shown in FIG. 9B (compared with FIG. 9A) may be an inherent product of the differential force multiplication. For example, at least initially, for a given length of wire that is pulled proximally through the tubular assembly, greater contraction and/or movement may be induced within the distal portion of the steering region (which has less mechanical advantage) than in the proximal portion of the steering region (which has greater mechanical advantage). Thus, due to its smaller mechanical advantage, the distal portion of the steering region may be more responsive (in terms of bending) than the proximal region. The overall effect may be that the steering region of FIG. 9B bends in a manner that more resembles rolling, whereas the steering region of FIG. 9A bends in a manner that more resembles deflection.

It is to be noted that the exemplary applications shown, e.g., in FIGS. 8B, 8D, 8E, and 8F, represent a non-exhaustive array of possible combinations of longitudinal segments, curved segments, and terminus positions for wire 140. For example, in some applications, wire 140 may have, e.g., three curved segments and four longitudinal segments, providing a greater mechanical advantage than that shown in FIG. 8B. Similarly, in some applications, terminus 158 may be disposed closer to the distal end of the steering region or closer to the proximal end of the steering region. Such arrangements may provide different bending behaviors and/or radii of curvature.

In some applications, the bending wire may be arranged to provide a greater mechanical advantage than the straightening wire. In some applications, the straightening wire may be arranged to provide a greater mechanical advantage than the bending wire. In some applications, the straightening wire may be arranged to provide no mechanical advantage, i.e., a force multiplication of one. In some applications, the bending wire may be arranged to provide no mechanical advantage, i.e., a force multiplication of one.

In some applications, a plurality of wires 140 (e.g., bending wires) may be arranged in parallel.

In some applications, a single wire 140 may be configured with multiple (e.g., two, three, or four) force-multiplication segments in series, each force-multiplication segment having a respective pulley arrangement having multiple longitudinal segments. This contrasts with the use of multiple wires to provide multiple force-multiplication segments in series, e.g., as described with reference to FIGS. 7A-B. Irrespective of how multiple in-series force-multiplication segments are provided, the multiple force-multiplication segments may each provide the same mechanical advantage as each other, or may provide different mechanical advantages to each other.

It is to be understood that, whereas the force-multiplication arrangements described with reference to FIGS. 8A-F relate to wire 140, which is described hereinabove as a bending wire, such force-multiplication arrangements may alternatively or additionally be applied, mutatis mutandis, to other wires such as a straightening wire, e.g., wire 180.

For some applications, the apparatus and techniques described herein, such as the tubular assemblies (e.g. catheters) and the arrangements of their pullwires, may be used in combination with those disclosed in International Patent Application PCT/IB2022/057505, filed Aug. 11, 2022, which is incorporated herein by reference. For example, the tubular assemblies (e.g. catheters) described herein may correspond to the tubes (e.g. catheters) described in PCT/IB2022/057505, and/or may be used to facilitate the techniques disclosed in PCT/IB2022/057505, mutatis mutandis.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the treatment techniques, methods, steps, etc. described or suggested herein or references incorporated herein can be performed on a living animal or on a non-living simulation, such as on a cadaver, cadaver heart, anthropomorphic ghost, simulator (e.g., with the body parts, tissue, etc. being simulated), etc.

1. Apparatus for facilitating a procedure on a subject, the apparatus comprising a catheter that comprises:
a head;
a tube comprising:
a proximal region, coupled to the head;
a steering region, distal from the proximal region, and dimensioned for advancement into the subject;
a bearing surface at the steering region; and
a circumferential wall extending from the proximal region to the steering region; and
a wire, wherein the wire:
extends, from the head, distally to the bearing surface,
is slidable over the bearing surface,
curves around the bearing surface and proximally away from the bearing surface, and
has a terminus that is anchored to the tube at an anchoring location.

2. The apparatus according to claim 1, wherein the wire defines:
a first longitudinal segment between the head and the bearing surface, and
a second longitudinal segment between the bearing surface and the terminus, and
wherein, within the steering region, the first longitudinal segment is substantially parallel with the second longitudinal segment.

3. The apparatus according to any one of claims 1-2, wherein the wire extends proximally away from the bearing surface to the terminus.

4. The apparatus according to any one of claims 1-3, wherein the steering region is a distal region of the tube.

5. The apparatus according to any one of claims 1-4, wherein, at the steering region, the wire is arranged in a force-multiplication arrangement that, upon application of a tensioning force to a proximal end of the wire, multiplies the tensioning force within the steering region to apply a bending force that bends the steering region.

6. The apparatus according to any one of claims 1-5, wherein the steering region is no more flexible than the proximal region.

7. The apparatus according to any one of claims 1-6, wherein the tube extends from the proximal region to the steering region to define a longitudinal axis of the tube, and in response to application of a tensioning force to a proximal end of the wire, at the bearing surface the wire slides around the longitudinal axis.

8. The apparatus according to any one of claims 1-7, wherein the tube extends from the proximal region to the steering region to define a longitudinal axis of the tube, and in response to application of a tensioning force to a proximal end of the wire, at the bearing surface the wire slides around a transverse axis that is transverse to the longitudinal axis.

9. The apparatus according to any one of claims 1-8, wherein the tube extends from the proximal region to the steering region to define a longitudinal axis of the tube, and in response to application of a tensioning force to a proximal end of the wire, at the bearing surface the wire slides around the longitudinal axis.

10. The apparatus according to any one of claims 1-9, wherein the tube is configured to be advanced through an anatomical lumen.

11. The apparatus according to any one of claims 1-10, wherein the tube is configured to be advanced through an airway duct.

12. The apparatus according to any one of claims 1-11, wherein the circumferential wall circumscribes, and thereby defines, a channel of the tube.

13. The apparatus according to claim 12, wherein the channel is configured to facilitate passage of a tool therethrough.

14. The apparatus according to claim 12, wherein the channel is configured to facilitate passage of an imaging device therethrough.

15. The apparatus according to claim 12, wherein the wire at least partially circumscribes the channel.

16. The apparatus according to claim 15, wherein, at the bearing surface, the wire circumscribes the channel in an arc of 330-360 degrees.

17. The apparatus according to claim 15, wherein, at the bearing surface, the wire circumscribes the channel in an arc of 330 degrees or less.

18. The apparatus according to claim 17, wherein, at the bearing surface, the wire circumscribes the channel in an arc of 180 degrees or less.

19. The apparatus according to claim 18, wherein, at the bearing surface, the wire circumscribes the channel in an arc of 90 degrees or less.

20. The apparatus according to any one of claims 1-19, wherein:
the bearing surface is a first bearing surface,
the tube comprises a second bearing surface, and
the wire:
extends, from the first bearing surface, proximally to the second bearing surface,
is slidable over the second bearing surface, and
curves around the second bearing surface and distally away from the second bearing surface.

21. The apparatus according to claim 20, wherein the wire extends distally to the terminus.

22. The apparatus according to claim 20, wherein the anchoring location is located adjacent the first bearing surface.

23. The apparatus according to claim 20, wherein the anchoring location is located adjacent the second bearing surface.

24. The apparatus according to claim 20, wherein the anchoring location is located partway between the first bearing surface and the second bearing surface.

25. The apparatus according to claim 24, wherein the anchoring location is located midway between the first bearing surface and the second bearing surface.

26. The apparatus according to claim 24, wherein the anchoring location is located closer to the first bearing surface than to the second bearing surface.

27. The apparatus according to claim 24, wherein the anchoring location is located closer to the second bearing surface than to the first bearing surface.

28. The apparatus according to claim 20, wherein:
the wire defines:
a first longitudinal segment between the head and the first bearing surface,
a second longitudinal segment between the first bearing surface and the second bearing surface, and
a third longitudinal segment between the second bearing surface and the terminus, and
within the steering region, the first longitudinal segment is substantially parallel with the second longitudinal segment.

29. The apparatus according to claim 28, wherein, within the steering region, the first longitudinal segment, the second longitudinal segment, and the third longitudinal segment are arranged in a force-multiplication arrangement.

30. The apparatus according to claim 28, wherein, within the steering region, the second longitudinal segment is substantially parallel with the third longitudinal segment.

31. The apparatus according to claim 28, wherein the third longitudinal segment terminates at the terminus.

32. The apparatus according to claim 28, wherein:
the steering region has a distal segment and a proximal segment,
the first and second longitudinal segments are present in the proximal segment and the distal segment, and
the third longitudinal segment is absent from the distal segment.

33. The apparatus according to claim 32, wherein the anchoring location is at a margin between the distal segment and the proximal segment.

34. The apparatus according to any one of claims 1-33, wherein the wire is a first wire, the terminus is a first terminus, and the catheter further comprises a second wire that extends from the head distally to a second terminus of the second wire, the second terminus being anchored to the tube.

35. The apparatus according to claim 34, wherein:
the first wire is engaged with the tube in a manner in which tensioning of the first wire bends the steering region; and
the second wire is engaged with the tube in a manner in which tensioning of the second wire straightens the steering region.

36. The apparatus according to claim 34, wherein, within the steering region:
the catheter comprises a shaft within the circumferential wall,
the first wire is mounted medially with respect to the shaft, and
the second wire is mounted laterally with respect to the shaft.

37. The apparatus according to claim 34, wherein the second wire is arranged in a force-multiplication arrangement.

38. The apparatus according to claim 37, wherein:
the force-multiplication arrangement is a second force-multiplication arrangement, the first wire being arranged in a first force-multiplication arrangement,
the first force-multiplication arrangement provides a first mechanical advantage, and
the second force-multiplication arrangement provides a second mechanical advantage that is different to the first mechanical advantage.

39. The apparatus according to any one of claims 1-38, wherein, within the steering region, the catheter comprises a shaft within the circumferential wall, the shaft being configured to facilitate steering of the steering region.

40. The apparatus according to claim 39, wherein the shaft is substantially tubular.

41. The apparatus according to claim 39, wherein the shaft comprises a chain of vertebrae mutually coupled at one side of the shaft and disjoined at another side of the shaft.

42. The apparatus according to claim 39, wherein the shaft comprises a chain of vertebrae, at least one of the vertebrae defining:
a pair of curved protrusions, disposed bilaterally on opposite sides of the vertebra, and
a pair of curved cavities, disposed bilaterally on opposite sides of the vertebra.

43. The apparatus according to claim 42, wherein adjacent vertebrae of the chain are coupled to each other via mating between the pairs of curved protrusions and the pairs of curved cavities.

44. The apparatus according to any one of claims 1-43, wherein the catheter comprises a support ring that provides the bearing surface.

45. The apparatus according to claim 44, wherein the bearing surface is a static bearing surface over which the wire is slidable.

46. The apparatus according to any one of claims 1-45, wherein the catheter comprises a rotatable bearing that provides the bearing surface.

47. The apparatus according to claim 46, wherein:
the tube extends from the proximal region to the steering region to define a longitudinal axis of the tube, and
the rotatable bearing is mounted in a manner that facilitates sliding of the wire around a transverse axis that is transverse to the longitudinal axis.

48. The apparatus according to claim 46, wherein:
the tube extends from the proximal region to the steering region to define a longitudinal axis of the tube, and
the rotatable bearing is mounted in a manner that facilitates sliding of the wire around the longitudinal axis.

49. The apparatus according to claim 46, wherein the circumferential wall circumscribes, and thereby defines, a channel of the tube, and the rotatable bearing circumscribes the channel in a manner that facilitates sliding of the wire around the channel.

50. The apparatus according to claim 46, wherein the rotatable bearing comprises a sheave.

51. The apparatus according to any one of claims 1-50, further comprising an extracorporeal interface.

52. The apparatus according to claim 51, wherein the extracorporeal interface is configured to receive the head of the catheter in a manner that operatively couples the extracorporeal interface to the steering region via the wire.

53. The apparatus according to claim 51, wherein the extracorporeal interface is configured to steer the steering region of the tube by applying a tensioning force to the first longitudinal segment.

54. An assembly for facilitating an endoscopic procedure on a subject, the assembly for use with an extracorporeal interface, and comprising:
a tube comprising:
a proximal region;
a steering region, distal from the proximal region, and dimensioned for advancement into the subject; and
a circumferential wall extending from the proximal region to the steering region; and
at least one wire engaged with the tube,
wherein for each wire of the at least one wire:
the wire has a first longitudinal segment coupled to the extracorporeal interface at the proximal region and extending distally therefrom to a curved segment of the wire positioned at a curvature site arranged at the steering region,
the curved segment curves at the curvature site and connects the first longitudinal segment to a second longitudinal segment of the wire,
the second longitudinal segment extends away from the curved segment, proximally along the steering region, and
the wire terminates at a terminus that is anchored to the tube at an anchoring location, the second longitudinal segment being positioned, along the wire, between the curved segment and the terminus.

55. The assembly according to claim 54, wherein the steering region is a first steering region of the tube, and wherein the tube further comprises:
at least one other steering region, disposed at a different axial location along the tube from the first steering region; and
for each of the other steering regions, at least one respective other wire engaged with the tube,
wherein, for each of the other wires:
the other wire has a first longitudinal segment coupled to the extracorporeal interface at the proximal region and extending distally therefrom to a curved segment of the other wire positioned at a curvature site arranged at the respective other steering region;
the curved segment curves at the curvature site and connects the first longitudinal segment to a second longitudinal segment of the other wire;
the second longitudinal segment extends away from the curved segment, proximally along the respective other steering region, and
the other wire terminates at a respective terminus that is anchored to the tube at a respective anchoring location, the second longitudinal segment being positioned, along the other wire, between the curved segment and the terminus.

56. The assembly according to claims 54-55, wherein the steering region is a distal region of the tube.

57. The assembly according to any one of claim 54-56, wherein, at the steering region, the wire is arranged in a force-multiplication arrangement that, upon application of a tensioning force to a proximal end of the wire, multiplies the tensioning force within the steering region to apply a bending force that bends the steering region.

58. The assembly according to any one of claims 54-57, wherein the steering region is no more flexible than the proximal region.

59. The assembly according to any one of claims 54-58, wherein the first longitudinal segment is substantially parallel with the second longitudinal segment.

60. The assembly according to any one of claims 54-59, wherein the tube extends from the proximal region to the steering region to define a longitudinal axis of the tube, and for each wire of the at least one wire, the wire is arranged such that, in response to tensioning of the wire, at the curvature site the wire slides around the longitudinal axis.

61. The assembly according to any one of claims 54-60, wherein the tube extends from the proximal region to the steering region to define a longitudinal axis of the tube, and for each wire of the at least one wire, the wire is arranged such that, in response to tensioning of the wire, at the curvature site the curved segment slides around a transverse axis that is transverse to the longitudinal axis.

62. The assembly according to any one of claims 54-61, wherein the tube is configured to be advanced through an anatomical lumen.

63. The assembly according to any one of claims 54-62, wherein the tube is configured to be advanced through an airway duct.

64. The assembly according to any one of claims 54-63, wherein the second longitudinal segment terminates at the terminus.

65. The assembly according to any one of claims 54-64, wherein, for at least one wire of the at least one wire:
the curvature site is a first curvature site,
the curved segment is a first curved segment, and
the second longitudinal segment extends proximally along the steering region to a second curved segment of the wire at a second curvature site.

66. The assembly according to claim 65, wherein the wire further defines:
a third longitudinal segment between the second curved segment and the terminus, and
within the steering region, the third longitudinal segment is substantially parallel with the second longitudinal segment.

67. The assembly according to claim 66, wherein, within the steering region, the first longitudinal segment, the second longitudinal segment, and the third longitudinal segment are arranged in a force-multiplication arrangement.

68. The assembly according to claim 66, wherein, for at least one wire of the at least one wire, the anchoring location is located closer to the first curvature site than to the second curvature site.

69. The assembly according to claim 66, wherein, for at least one wire of the at least one wire, the anchoring location is located closer to the second curvature site than to the first curvature site.

70. The assembly according to claim 66, wherein the anchoring location is located adjacent the first curvature site.

71. The assembly according to claim 66, wherein the anchoring location is located adjacent the second curvature site.

72. The assembly according to claim 66, wherein, the anchoring location is located partway between the first curved segment and the second curved segment.

73. The assembly according to claim 72, wherein, the anchoring location is located midway between the first curved segment and the second curved segment.

74. The assembly according to claim 66, wherein the at least one wire extends distally to the terminus.

75. The assembly according to any one of claims 54-65, wherein the circumferential wall circumscribes, and thereby defines, a channel of the tube.

76. The assembly according to claim 75, wherein the channel is configured to facilitate passage of a tool therethrough.

77. The assembly according to claim 75, wherein the channel is configured to facilitate passage of an imaging device therethrough.

78. The assembly according to claim 75, wherein the curved segment at least partially circumscribes the channel at the curvature site.

79. The assembly according to claim 78, wherein the second longitudinal segment extends to a second curved segment of the wire, the second curved segment at least partly circumscribing the channel.

80. The assembly according to claim 78, wherein the curved segment circumscribes the channel in an arc of 330-360 degrees.

81. The assembly according to claim 78, wherein the curved segment circumscribes the channel in an arc of 330 degrees or less.

82. The assembly according to claim 78, wherein the curved segment circumscribes the channel in an arc of 180 degrees or less.

83. The assembly according to claim 78, wherein the curved segment circumscribes the channel in an arc of 90 degrees or less.

84. The assembly according to any one of claims 54-83, wherein the at least one wire comprises:
a first wire, engaged with the tube in a manner in which tensioning of the first wire bends the steering region; and
a second wire, engaged with the tube in a manner in which tensioning of the second wire straightens the steering region.

85. The assembly according to claim 84, wherein the first wire comprises a first-wire curved segment and the second wire comprises a second-wire curved segment, the second-wire curved segment being disposed proximally from the first-wire curved segment.

86. The assembly according to claim 84, wherein:
within the steering region, the tube comprises a shaft within the circumferential wall,
the first wire is mounted medially with respect to the shaft, and
the second wire is mounted laterally with respect to the shaft.

87. The assembly according to claim 79, further including a third longitudinal segment of the wire which extends from the second curved segment and terminates at the terminus.

88. The assembly according to claim 87, wherein:
the steering region comprises a distal part and a proximal part; and
the first and third longitudinal segments extend from the proximal part to the distal part.

89. The assembly according to claim 88, wherein the second longitudinal segment extends from the distal part to the proximal part.

90. The assembly according to claim 89, wherein the terminus is positioned at the distal part.

91. The assembly according to claim 89, wherein the terminus is positioned at the proximal part.

92. The assembly according to claim 89, wherein the terminus is positioned intermediate the distal part and the proximal part.

93. The assembly according to any one of claims 54-92, wherein, within the steering region, the tube comprises a shaft within the circumferential wall, the shaft being configured to facilitate steering of the steering region.

94. The assembly according to claim 93, wherein the shaft is substantially tubular.

95. The assembly according to claim 93, wherein the shaft comprises a chain of vertebrae mutually coupled at one side of the shaft and disjoined at another side of the shaft.

96. The assembly according to claim 95, wherein at least one of the vertebrae defines:
a pair of curved protrusions, disposed bilaterally on opposite sides of the vertebra, and
a pair of curved cavities, disposed bilaterally on opposite sides of the vertebra.

97. The assembly according to claim 96, wherein adjacent vertebrae of the chain are coupled to each other via mating between the pairs of curved protrusions and the pairs of curved cavities.

98. The assembly according to any one of claims 54-97, wherein the tube comprises a support ring on which the curved segment is mounted.

99. The assembly according to claim 98, wherein the support ring defines a static bearing surface over which the curved segment is slidable.

100. The assembly according to any one of claims 54-99, wherein the tube comprises a rotatable bearing mounted on the steering region and the wire is configured to move around the rotatable bearing.

101. The assembly according to claim 100, wherein:
the tube extends from the proximal region to the steering region to define a longitudinal axis of the tube, and
the rotatable bearing is mounted in a manner that facilitates sliding of the wire around a transverse axis that is transverse to the longitudinal axis.

102. The assembly according to claim 100, wherein:
the tube extends from the proximal region to the steering region to define a longitudinal axis of the tube, and
the rotatable bearing is mounted in a manner that facilitates sliding of the wire around the longitudinal axis.

103. The assembly according to claim 100, wherein the circumferential wall circumscribes, and thereby defines, a channel of the tube, and the rotatable bearing circumscribes the channel in a manner that facilitates sliding of the wire around the channel.

104. The assembly according to claim 100, wherein the rotatable bearing comprises a sheave.

105. The assembly according to any one of claims 54-104, further comprising the extracorporeal interface.

106. The assembly according to claim 105, wherein the extracorporeal interface is configured to receive the proximal region of the tube in a manner that operatively couples the extracorporeal interface to the steering region via the at least one wire.

107. The assembly according to claim 105, wherein the extracorporeal interface is configured to steer the steering region of the tube by applying a tensioning force to the first longitudinal segment.

108. The assembly according to any one of claims 54-107, wherein the first longitudinal segment, the curved segment, the second longitudinal segment, and terminus are arranged in a force-multiplication arrangement within the steering region.

109. An assembly for facilitating a procedure on a subject, the assembly for use with an extracorporeal interface, and comprising a catheter that comprises:
a proximal region;
a steering region, distal to the proximal region, and dimensioned for advancement into the subject;
a circumferential wall extending from the proximal region to the steering region; and
a wire, extending distally along the circumferential wall from the proximal region to the steering region,
wherein, at the steering region, the assembly is configured to define a force-multiplication arrangement that, upon application of a tensioning force to a proximal end of the wire, multiplies the tensioning force within the steering region to apply a bending force that bends the steering region.

110. The assembly according to claim 109, wherein the force-multiplication arrangement is a first force-multiplication arrangement, and wherein the wire is further arranged in a second force-multiplication arrangement that is configured to multiply the tensioning force by a different factor compared with the first force-multiplication arrangement.

111. The assembly according to any one of claims 109-110, wherein:
the wire has a first longitudinal segment coupled to the extracorporeal interface at the proximal region and extending distally therefrom to a curved segment of the wire positioned at a curvature site arranged at the steering region,
the curved segment curves at the curvature site and connects the first longitudinal segment to a second longitudinal segment of the wire,
the second longitudinal segment extends away from the curved segment, proximally along the steering region,
the wire terminates at a terminus that is anchored to the catheter at an anchoring location, the second longitudinal segment being positioned, along the wire, between the curved segment and the terminus, and
the wire is arranged for force multiplication of the applied tensioning force by curving the curved segment at the curvature site, thereby subjecting each one of the first and second longitudinal segments with a tensioning force equivalent to the applied tensioning force.

112. The assembly according to any one of claims 109-111, wherein:
the steering region is a first steering region,
the at least one wire is at least one first wire,
the catheter comprises:
a second steering region, proximal from the first steering region, and
a second wire engaged with the catheter,
from the proximal end of the first wire, the first wire extends distally along the circumferential wall, past the first steering region to the second steering region, and
at the second steering region, the assembly is configured to define a second force-multiplication arrangement that, upon application of a tensioning force to a proximal end of the second wire, multiplies the tensioning force within the second steering region to apply a bending force that bends the second steering region.

113. The assembly according to any one of claims 109-112, wherein the wire extends proximally to the terminus.

114. The assembly according to any one of claims 109-113, wherein the wire extends distally to the terminus.

115. The assembly according to any one of claims 109-114, wherein the force-multiplication arrangement is configured to define a distal segment of the steering region and a proximal segment of the steering region, and to multiply the tensioning force by a different factor in the distal segment compared with in the proximal segment.

116. The assembly according to any one of claims 109-115, wherein the wire is a first wire, and wherein the assembly further comprises a second wire.

117. The assembly according to claim 116, wherein the second wire extends distally to a terminus that is anchored to the tube.

118. The assembly according to claims 116, wherein the force-multiplication arrangement is a first force-multiplication arrangement, and wherein the second wire is arranged in a second force-multiplication arrangement that is configured to multiply the tensioning force by a different factor in the distal segment compared with in the proximal segment.

119. An assembly for facilitating an endoscopic procedure on a subject, the assembly for use with an extracorporeal interface, and comprising:
a tube comprising:
a proximal region;
a steering region, distal from the proximal region, and dimensioned for advancement into the subject; and
a circumferential wall circumscribing a longitudinal axis of the tube, and
extending from the proximal region to the steering region along the longitudinal axis;
a first wire, engaged with the tube, and defining a first-wire longitudinal segment extending, within the circumferential wall, along the steering region; and
a second wire, engaged with the tube, and defining a second-wire longitudinal segment extending, within the circumferential wall, along the steering region,
wherein, in the steering region, the second-wire longitudinal segment is disposed more laterally than is the first-wire longitudinal segment.

120. The assembly according to claim 119, wherein the first-wire longitudinal segment is substantially parallel with the second-wire longitudinal segment.

121. The assembly according to any one of claims 119-120, wherein the first-wire and second-wire longitudinal segments are substantially parallel with the longitudinal axis.

122. The assembly according to any one of claims 119-121, wherein the first wire and the second wire are wires operable for steering the steering region for advancement of the tube into the subject.

123. The assembly according to any one of claims 119-122, wherein, for each of the first wire and the second wire:
the wire has a first longitudinal segment coupled to the extracorporeal interface at the proximal region and extending distally therefrom to a curved segment of the wire positioned at a curvature site arranged at the steering region,
the curved segment curves at the curvature site and connects the first longitudinal segment to a second longitudinal segment of the wire, and
the second longitudinal segment extends away from the curved segment, proximally along the steering region, and terminates at a terminus that is anchored to the tube at an anchoring location, the second longitudinal segment being positioned, along the wire, between the curved segment and the terminus.

124. The assembly according to any one of claims 119-123, wherein the first wire is configured for bending at least the steering region and the second wire is configured for straightening at least the steering region.

125. The assembly according to any one of claims 119-124, wherein:
the first wire is configured for bending at least the steering region and is longitudinally arranged to extend at least partially along an internal surface of the circumferential wall; and
the second wire is configured for straightening at least the steering region and is longitudinally arranged to extend at least partially along an external surface of the circumferential wall.

126. The assembly according to any one of claims 119-125, wherein the circumferential wall circumscribes, and thereby defines, a channel of the tube.

127. The assembly according to claim 126, wherein the first-wire longitudinal segment and second-wire longitudinal segment are disposed at opposite sides of the channel.

128. The assembly according to any one of claims 119-127, wherein the steering region comprises a tubular shaft within the circumferential wall, the tubular shaft formed with a circumferential shaft wall.

129. The assembly according to claim 128, wherein the circumferential wall comprises a flexible polymer and the tubular shaft is embedded within the flexible polymer.

130. The assembly according to claim 128, wherein the first-wire longitudinal segment and second-wire longitudinal segment are disposed at opposite sides of the circumferential shaft wall.

131. The assembly according to claim 128, wherein the circumferential shaft wall defines an internal surface and an external surface and first-wire longitudinal segment, and second-wire longitudinal segment are disposed at opposite sides of the circumferential shaft wall.

132. The assembly according to claim 131, wherein tensioning of the first wire presses the first wire against the internal surface of the circumferential shaft wall and tensioning of the second wire presses the second wire against the external surface of the circumferential shaft wall.

133. A method for use with an anatomical lumen of a subject, the method comprising:
advancing, towards the anatomical lumen, an assembly that includes:
a tube, having a proximal region, and a steering region distal from the proximal region, and
a wire, having a proximal end, extending along the tube to the steering region, and arranged within the steering region to define a force-multiplication arrangement; and
bending the steering region by applying a tensioning force to the proximal end of the wire such that the tensioning force is multiplied, within the steering region, by the force-multiplication arrangement.

134. The method according to claim 133, further comprising multiplying the tensioning force by providing the wire with the force-multiplication arrangement at the steering region, thereby steering the assembly by use of a resultant multiplied tensioning force, wherein a magnitude of the tensioning force required for steering the tube equipped with the force-multiplication arrangement at the steering region is less than that required for steering a comparable tube that is unequipped with the force-multiplication arrangement.

135. The method according to any one of claims 133-134, wherein:
the wire includes a first longitudinal segment extending to a curved segment of the wire positioned at a curvature site, the curved segment curving at the curvature site and connecting the first longitudinal segment to a second longitudinal segment of the wire, and
multiplying the tensioning force comprises multiplying the tensioning force facilitated by at least one of the curved segment, the first longitudinal segment, and the second longitudinal segment.

136. The method according to any one of claims 133-135, wherein the assembly includes a bearing at the steering region, and wherein bending the steering region comprises bending the steering region by applying the tensioning force to the proximal end of the wire such that the wire slides around the bearing.

137. The method according to claim 136, wherein the bearing is a rotatable bearing, and wherein bending the steering region comprises bending the steering region by applying the tensioning force to the proximal end of the wire such that the wire slides around the rotatable bearing.
